(19) United States
(12) Patent Application Publication
(10) Pub. No.: US 2004/0253304 A1
(43) Pub. Date: Dec. 16, 2004

(54) Active drug delivery in the gastrointestinal tract

(76) Inventors: Yossi Gross, Moshav Mazor (IL); Yoram Sela, Ra'anana (IL)

Correspondence Address: RATNERPRESTIA, P O BOX 980, VALLEY FORGE, PA, 19482-0980, US

(21) Appl. No.: 10/767,663

(22) Filed: Jan. 29, 2004

Related U.S. Application Data
(60) Provisional application No. 60/443,173, filed on Jan. 29, 2003.

(51) Int. Cl. A61K 9/48

(52) U.S. Cl. 424/451

(57) ABSTRACT

Apparatus for drug administration is provided, including an ingestible capsule. The capsule includes a drug, stored by the capsule. An environmentally-sensitive mechanism is adapted to change a state thereof responsive to a disposition of the capsule within a gastrointestinal tract of a subject. A driving mechanism, in response to a change of state of the environmentally-sensitive mechanism, is adapted to drive the drug directly through an endothelial layer of the gastrointestinal tract.

### CROSS-REFERENCES TO RELATED APPLICATIONS

This application claims the benefit of U.S. Provisional Patent Application 60/443,173, filed Jan. 29, 2003, which is incorporated herein by reference.

### FIELD OF THE INVENTION

The present invention relates to an oral drug delivery system and, more particularly, to an ingestible capsule which acts as a medication carrier and which enhances the absorption of the medication through the gastrointestinal wall.

### BACKGROUND OF THE INVENTION

The absorption of a drug (or of a drug precursor) into the systemic circulation is determined by the physicochemical properties of the drug, its formulations, and the route of administration, whether oral, rectal, topical, by inhalation, or by intravenous administration. Oral administration includes swallowing, chewing, sucking, as well as buccal administration, i.e., placing a drug between the gums and cheek, and sublingual administration, i.e., placing a drug under the tongue. A prerequisite to absorption is drug dissolution.

Absorption of orally-administered drugs into the internal environment generally occurs almost exclusively in the small intestine. The small intestine is lined with a layer of epithelial cells joined by tight junctions. In order to pass from the lumen of the small intestine into the internal environment and, therefrom into the systemic circulation, a dissolved drug must either pass through the semi-permeable membranes of the epithelial cells (transcellular passage), or through the tight junctions between the epithelial cells. The rate of transcellular passage is generally low except for small, lipid-soluble molecules. In addition, the tight junctions generally prevent the passage of most dissolved molecules. A drug may cross the biological barrier by passive diffusion, or by other naturally-occurring transfer modes, for example, facilitated passive diffusion, active transport, or pinocytosis. Alternatively, a drug may be artificially assisted to cross the biological barrier.

In passive diffusion, transport depends on the concentration gradient of the solute across the biological barrier. Since the drug molecules are rapidly removed by the systemic circulation, drug concentration in the blood in the vicinity of the administration site is low compared with that at the administration site, producing a large concentration gradient. The drug diffusion rate is directly proportional to that gradient. The drug diffusion rate also depends on other parameters, for example, the molecule's lipid solubility and size. Because the cell membrane is lipoid, lipid-soluble drugs diffuse more rapidly than relatively lipid-insoluble drugs. Similarly, small drug molecules penetrate biological barriers more rapidly than large ones.

Another naturally occurring transfer mode is facilitated passive diffusion, which occurs for certain molecules, such as glucose. It is believed that a carrier component combines reversibly with a substrate molecule at the cell membrane exterior. The carrier-substrate complex diffuses rapidly across the membrane, releasing the substrate at the interior surface. This process is characterized by selectivity and saturability: The carrier is operative only for substrates with a relatively specific molecular configuration, and the process is limited by the availability of carriers.

Active transport, which is another naturally occurring transfer mode, appears to be limited to drugs that are structurally similar to endogenous substances. Active transport is characterized by selectivity and saturability and requires energy expenditure by the cell. It has been identified for various ions, vitamins, sugars, and amino acids.

Still another naturally occurring transfer mode is pinocytosis, in which fluids or particles are engulfed by a cell. The cell membrane encloses the fluid or particles, then fuses again, forming a vesicle that later detaches and moves to the cell interior. Like active transport, this mechanism requires energy expenditure. It is known to play a role in drug transport of protein drugs.

The foregoing discussion relates to naturally occurring transfer modes. Where these are insufficient, for example, in cases of macromolecules and polar compounds, which cannot effectively traverse the biological barrier, drug transport may be artificially induced.

Electrotransport refers generally to electrically induced passage of a drug (or a drug precursor) through a biological barrier. Several electrotransport mechanisms are known, as follows:

Iontophoresis involves the electrically induced transport of charged ions, by the application of low-level, direct current (DC) to a solution of the medication. Since like electrical charges repel, the application of a positive current drives positively charged drug molecules away from the electrode and into the tissues; similarly, a negative current will drive negatively charge ions into the tissues. Iontophoresis is an effective and rapid method of delivering water-soluble, ionized medication. Where the drug molecule itself is not water-soluble, it may be coated with a coating (for example, sodium lauryl sulfate (SLS)), that may form water-soluble entities.

Electroosmosis involves the movement of a solvent with the agent through a membrane under the influence of an electric field.

Electrophoresis is based on migration of charged species in an electromagnetic field. Ions, molecules, and particles with charge carry current in solutions when an electromagnetic field is imposed. Movement of a charged species tends to be toward the electrode of opposite charge. The voltages for continuous electrophoresis are rather high (several hundred volts).

Electroporation is a process in which a biological barrier is subjected to a high-voltage alternating-current (AC) surge, or pulse. The AC pulse creates temporary pores in the biological membrane, specifically between cells. The pores allow large molecules, such as proteins, DNA, RNA, and plasmids to pass through the biological barrier.

Iontophoresis, electroosmosis, and electrophoresis are diffusion processes, in which diffusion is enhanced by electrical or electromagnetic driving forces. In contrast, electroporation physically punctures the biological barriers, along cell boundaries, enabling passage of large molecules through the epithelium.

Generally, during electrotransport a combination of more than one of these processes occurs, together with passive diffusion and other naturally-occurring transfer modes. Therefore, electrotransport refers to at least one, and possibly a combination of the aforementioned transport mechanisms, which supplement the naturally-occurring transfer modes. Medical devices that include drug delivery by electrotransport are described, for example, in U.S. Pat. No. 5,674,196 to Donaldson et al., U.S. Pat. No. 5,961,482 to Chien et al., U.S. Pat. No. 5,983,131 to Weaver et al., U.S. Pat. No. 5,983,134 to Ostrow, and U.S. Pat. No. 6,477,410 to Henley et al., all of whose disclosures are incorporated herein by reference.

In addition to the aforementioned electrotransport processes, there are other electrically assisted drug delivery mechanisms, including:

Sonophoresis, i.e., the application of ultrasound, induces growth and oscillations of air pockets, a phenomenon known as cavitation. These disorganize lipid bilayers thereby enhancing transport. For effective drug transport, a low frequency of between 20 kHz and less than 1 MHz, rather than the therapeutic frequency, should be used. Sonophoresis devices are described, for example, in U.S. Pat. Nos. 6,002,961, 6,018,678, and 6,002,961 to Mitragotri et al., U.S. Pat. Nos. 6,190,315 and 6,041,253 to Kost et al., U.S. Pat. No. 5,947,921 to Johnson et al., and U.S. Pat. Nos. 6,491,657 and 6,234,990 to Rowe et al., all of whose disclosures are incorporated herein by reference.

Ablation is another method of facilitating drug passage through a biological barrier. In addition to mechanical ablation, for example using hypodermic needles, ablation techniques include laser ablation, cryogenic ablation, thermal ablation, microwave ablation, radiofrequency ablation, liquid jet ablation, or electrical ablation.

U.S. Pat. No. 6,471,696 to Berube et al. describes a microwave ablation catheter, which may be used as a drug delivery device. U.S. Pat. No. 6,443,945 to Marchitto et al. describes a device for pharmaceutical delivery using laser ablation. U.S. Pat. No. 4,869,248 to Narula describes a catheter for performing localized thermal ablation, for purposes of drug administration. U.S. Pat. Nos. 6,148,232 and 5,983,135 to Avrahami describe drug delivery systems using electrical ablation. The disclosures of all of these patents are incorporated herein by reference.

Oral drug administration is a common drug delivery route. Drug bioavailability of orally administered drugs, i.e., the degree to which the drug is available to the target tissue, is affected by drug dissolution, drug degradation in the gastrointestinal (GI) tract, and drug absorption.

Drug dissolution is affected by whether the drug is in salt, crystal, or hydrate form. To improve dissolution, disintegrants and other excipients, such as diluents, lubricants, surfactants (substances which increase the dissolution rate by increasing the wettability, solubility, and dispersibility of the drug), binders, or dispersants are often added during manufacture.

Drug degradation in the GI tract is due to GI secretions, low pH values, and degrading enzymes. Since luminal pH varies along the GI tract, the drug must withstand different pH values. Interaction with blood, food staff, mucus, and bile may also affect the drug. Reactions that may affect the drug, and reduce bioavailability, include: (a) complex formations, for example, between tetracycline and polyvalent metal ions; (b) hydrolysis by gastric acid or digestive enzymes, for example, penicillin and chloramphenicol palmitate hydrolysis; (c) conjugation in the gut wall, for example, sulfoconjugation of isoproterenol; (d) adsorption to other drugs, for example, digoxin and cholestyramine; and (e) metabolism by luminal microflora.

Drug absorption of orally-administered drugs relates to transport of drugs across biological barriers presented by the epithelial cells in the GI tract. The nature of intestinal epithelium tends to inhibit drug absorption. As seen in FIG. 1 (based on Martinit, F. H., et al., Human Anatomy, Prentice Hall, Englewood Cliffs, N.J., 1995), the intestinal epithelium of the small intestine is formed as a series of finger-like projections, called intestinal villi. These are covered by columnar epithelium, carpeted with microvilli. The epithelial cells along the microvilli are strongly bound to each other, by tight junctions, also called the zona occludens. The tight junctions seal the internal environment of the body from the intestinal lumen. The size of gaps between tight junctions in humans is about 8 nm in the jejunum, and about 0.3 nm in the ileum and the colon. Therefore, particles with diameters greater than about 11.5 angstrom and/or several thousand daltons generally cannot penetrate the gaps.

Overall, low bioavailability is most common with oral dosage forms of poorly water-soluble, slowly absorbed drugs. Insufficient time in the GI tract is another common cause of low bioavailability. An ingested drug is exposed to the entire GI tract for no more than 1 to 2 days, and to the small intestine for only about 2 to 4 hours. If the drug does not dissolve readily or cannot penetrate the epithelial membrane quickly, its bioavailability will be low. Age, sex, activity, genetic phenotype, stress, disease (e.g., achlorhydria, malabsorption syndromes), or previous GI surgery can further affect drug bioavailability.

Table 1 below (from Encyclopedia of Controlled Drug Delivery, edited by Edith Mathiowitz) summarizes some parameters of the oral route that affect drug bioavailability.

TABLE 1

| | | Liquid | | Transit |
|---|---|---|---|---|
| | Area, | Secretion, | pH | Time, |
| Section | m² | liters/day | Value | hours |
| Oral | ˜0.05 | 0.5-2 | 5.2-6.8 | Short |
| cavity | | | | |
| Stomach | 0.1-0.2 | 2-4 | 1.2-3.5 | 1-2 |
| Duodenum | ˜0.04 | 1-2 | 4.6-6.0 | 1-2 |
| Small | 4500 | 0.2 | 4.7-6.5 | 1-10 |
| Intestine | (including | | | |
| | microvilli) | | | |
| Large | 0.5-1 | ˜0.2 | 7.5-8.0 | 4-20 |
| Intestine | | | | |

In addition to the physical barrier of the epithelial cells, chemical and enzymatic barriers affect drug absorption.

It is known to provide an ingestible capsule that includes a drug and a chemical that indirectly facilitates passage of the drug across the endothelial layer. For example, the chemical may induce a change in the endothelial layer that renders it transiently more permeable to the drug, whereupon the drug (indirectly facilitated by the action of the chemical), crosses the endothelial layer by diffusion.

Another important barrier to drug absorption is the pre-systematic, first-pass metabolism, primarily hepatic metabolism. The predominant enzymes in this metabolism are the multi-gene families of cytochrome P450, which have a central role in metabolizing drugs. It appears that variations in P450s between individuals lead to variations in their ability to metabolize the same drug.

Additionally, multidrug resistance (MDR) may be a barrier to drug absorption. MDR, which is a major cause of cancer treatment failure, is a phenomenon whereby cancer cells develop a broad resistance to a wide variety of chemotherapeutic drugs. MDR has been associated with overexpression of P-glycoprotein or multidrug resistance-associated protein (MRP), two transmembrane transporter molecules which act as pumps to remove toxic drugs from tumor cells. P-glycoprotein acts as a unidirectional efflux pump in the membrane of acute myeloid leukemia (AML) cells and lowers the intracellular concentration of cytotoxic agents, by pumping them out of leukemic cells. Yet it confers resistance to a variety of chemotherapy drugs, including daunorubicin.

Ingestible radio pills, which are ingestible capsules containing a transmitter and other electrical components are known. In 1964 researchers at Heidelberg University developed a pill for monitoring pH of the GI tract. (Noller, H. G., “The Heidelberg Capsule Used For the Diagnosis of Peptic Diseases,” Aerospace Medicine, February, 1964, pp. 115-117.)

U.S. Pat. No. 4,844,076 to Lesho et al., issued July 1989, entitled, “Ingestible size continuously transmitting temperature monitoring pill,” whose disclosure is incorporated herein by reference, describes a temperature responsive transmitter, encapsulated in an ingestible size capsule. The capsule is configured to monitor average body temperature, internally. The ingestible size temperature pill can be configured in a rechargeable embodiment. In this embodiment the pill uses the inductive coil in the tank circuit as the magnetic pickup to charge a rechargeable nickel cadmium battery.

U.S. Pat. No. 5,279,607 to Schentag et al., entitled, “Telemetry capsule and process,” whose disclosure is incorporated herein by reference, describes an ingestible capsule and a process for delivery, particularly repeatable delivery, of a medicament to the alimentary canal. The ingestible capsule is an essentially non-digestible capsule, which contains an electric energy emitting means, a radio signal transmitting means, a medicament storage means and a remote actuatable medicament releasing means. The capsule signals a remote receiver as it progresses through the alimentary tract in a previously mapped route and upon reaching a specified site is remotely triggered to release a dosage of medicament.

U.S. Pat. No. 5,395,366 to D'Andrea et al., entitled, “Sampling capsule and process,” whose disclosure is incorporated herein by reference, describes a similar ingestible capsule and a process for sampling of fluids in the alimentary canal.

The use of electrostimulating capsules for promoting peristalsis is known. PCT Publications WO 97/31679 to Dirin and WO 97/26042 to Terekhin, the disclosures of both of which are incorporated herein by reference, disclose ingestible capsules for electrostimulation of the alimentary tract, to be used, for example, as a post-surgical therapy, as a prophylactic measure of alimentary tract diseases, or for the promotion of peristalsis.

PCT Publication WO 97/31679 further discloses that USSR Inventor's Certificate No. 1223922, Int. Cl. A 61 N 1/36, Bulletin No. 14, by Pekarasky et al., entitled, “Gastrointestinal tract Electrostimulator,” which is incorporated herein by reference, describes a swallowable capsule adapted for electrostimulation of the alimentary tract, as post-surgical therapy, as a prophylactic measure of alimentary tract diseases, or for the promotion of peristalsis, which is further adapted for the dispensing of medication.

Methods of tracking ingestible devices, such as radio pills, are described, for example, in the above-mentioned U.S. Pat. No. 5,279,607 to Schentag et al., the above-mentioned U.S. Pat. No. 5,395,366 to D'Andrea et al., and U.S. Pat. No. 6,082,366 to Andrii et al., entitled, “Method and arrangement for determining the position of a marker in an organic cavity,” all of whose disclosures are incorporated herein by reference.

Visual examination of the GI tract by ingestible devices is known. U.S. Pat. No. 5,984,860 to Shan, entitled, “Pass-through duodenal enteroscopic device,” whose disclosure is incorporated herein by reference, describes a tethered ingestible, enteroscopic video camera, which utilizes the natural contraction wave of the small intestine to propel it through the small intestine at about the same speed as any other object therein. The video camera includes an illumination source at its forward end. Covering the camera lens and illumination source is a transparent inflatable balloon, adapted to gently expand the small intestine immediately forward the camera for better viewing. A small diameter communication and power cable unwinds through an aperture in the rear of the camera as it moves through the small intestine. Upon completion of movement through the small intestine the cable is automatically separated, permitting the cable to be withdrawn through the stomach and intestine. The camera continues through the large intestine and passes from the patient through the rectum.

U.S. Pat. No. 5,604,531 to Iddan et al., entitled, “In vivo video camera system,” whose disclosure is incorporated herein by reference, describes a video camera system, encapsulated within an ingestible capsule, arranged to pass through the entire digestive tract, operating as an autonomous video endoscope. The ingestible capsule includes a camera system and an optical system for imaging an area of interest onto the camera system, and a transmitter, which relays the video output of the camera system to an extracorporeal reception system. A light source is located within a borehole of the optical system.

Similarly, US Patent Application 2001/0035902 to Iddan et al., entitled, “Device and system for in vivo imaging,” whose disclosure is incorporated herein by reference, describes a system and method for obtaining in vivo images. The system contains an imaging system and an ultra low power radio frequency transmitter for transmitting signals from a CMOS imaging camera to a receiving system located outside a patient.

Additionally, U.S. Pat. No. 6,428,469 to Iddan et al., entitled, “Energy management of a video capsule,” whose disclosure is incorporated herein by reference, describes an energy saving device for acquiring in vivo images of the gastro-intestinal tract. The device, such as an autonomous capsule, includes at least one imaging unit, a control unit connected to the imaging unit, and a power supply connected to the control unit. The control unit includes a switching unit, and an axial motion detector connected to the switching unit, which disconnects the power supply thereby preventing the acquisition of redundant images.

U.S. Pat. No. 6,632,216 to Houzego et al., which is incorporated herein by reference, describes an ingestible device for delivering a substance to a chosen location in the GI tract. The device includes a receiver of electromagnetic radiation for powering an openable part of the device to an opened position for dispensing of the substance. The receiver includes a coiled wire that couples the energy field, the wire having an air or ferrite core. The device optionally includes a latch defined by a heating resistor and a fusible restraint. The device may also include a flexible member that may serve one or both the functions of activating a transmitter circuit to indicate dispensing of the substance, and restraining of a piston used for expelling the substance.

PCT Publication WO 02/094369 to Walla, which is incorporated herein by reference, describes a device for applying substances such as medicaments having a liquid, ointment or gel-like consistency through the skin, especially by means of iontophoresis. The resorption of the substance occurs by application of a DC current. The publication also describes a capsular, hermetically sealed container for insertion into body orifices, which has at least two electrodes for generating a continuous electric field on its outer side. A device for receiving the substance to be applied is provided above the electrodes. The container is positioned to be in contact with the mucous membrane and/or the skin in a body orifice, especially in the urogenital, vaginal, and/or anal tract, and/or in the cavities of the mouth, ear, and/or nose.

U.S. Pat. No. 5,217,449 to Yuda et al., which is incorporated herein by reference, describes a capsule having an outer cylinder and a piston movable in the outer cylinder, the piston being activated by an externally given signal so as to discharge a medicine to the outside of the capsule or to suck a humor for a sampling purpose. The capsule has a remote-controllable means including a normally-opened lead switch which connects a power supply to an activating means in response to an externally given magnetic signal thereby initiating activation of the capsule.

U.S. Pat. No. 5,464,395 to Faxon et al., which is incorporated herein by reference, describes a catheter for delivering therapeutic and/or diagnostic agents directly into the tissue surrounding a bodily passageway. The catheter comprises at least one needle cannula able to be projected outboard of the catheter so as to deliver the desired agents to the tissue. The catheter also preferably includes one or more inflatable balloons.

U.S. Pat. No. 4,239,040 to Hosoya et al., which is incorporated herein by reference, describes a capsule for discharging drugs into a body or collecting samples from the body. The capsule comprises an external cylinder having slidably mounted therein an internal cylinder. The internal cylinder is retained by a meltable thread at one end of the external cylinder against the biasing force of a compression spring. Upon melting of the thread, the spring effects sliding of the internal cylinder to the other end of the external cylinder, and, during this sliding movement, a drug is pushed out of the external cylinder ahead of the moving internal cylinder or a body sample is withdrawn into the external cylinder behind the moving internal cylinder. An electric circuit including a tunable receiver responds to an externally-transmitted electric signal to energize a heater for melting the thread to thereby effect sliding movement of the internal cylinder at the desired time.

U.S. Pat. No. 4,425,117 to Hugemann et al., which is incorporated herein by reference, describes a capsule for the release of a substance at a defined or desired location in the alimentary tract. The capsule has a separating wall therein, which forms a first chamber and a second chamber, the first chamber having a hole in a wall thereof. A compression spring, in a compressed state, is affixed to a body located in the second chamber. A needle is mounted on the compression spring facing the separation wall. A resonant circuit in the second chamber is tuned to an electromagnetic field of high frequency. The resonant circuit has a coupling coil, positioned around the body, a capacitor, connected to the other end of the coil and extending away from the first chamber, and a resistance wire, attached to the coupling coil and the capacitor. A fuse wire is connected to the compression spring, extends through the longitudinal passageway of the body and is connected to the body end facing away from the first chamber. The fuse wire contacts the resistance wire. A balloon in the expanded state is positioned in the first chamber. When the device is subjected to an external electromagnetic field having the high frequency to which the resonant circuit is tuned, the fuse wire heats up and breaks. The compressed spring is released pushing the point of the needle through the separating wall and the balloon, which bursts releasing any substance contained in the first chamber.

U.S. Pat. No. 4,507,115 to Kambara et al., which is incorporated herein by reference, describes a capsule that comprises a capsule body having a chamber formed inside and a communicating path for communicating the chamber with outside, a movable member arranged in the chamber and movable between a liquid-receiving position at which the volume of said chamber is made largest and a liquid-pushing position at which the volume of said chamber is made smallest, and a coiled operating member made of shape memory alloy heated by ultrasonic wave to move the movable member to liquid-receiving and -pushing positions selectively.

U.S. Pat. No. 5,951,538 to Joshi et al., which is incorporated herein by reference, describes a controlled delivery device for holding and administering a biologically active agent. The device includes a housing having a first end portion, a second end portion, and a port associated with the housing. Enclosed within the housing is a displacing member, a chemical or electrochemical gas generating cell, and activation and control circuitry. The electrochemical or chemical cell generates gas within the housing, forcing the displacing member against the beneficial agents contained within the housing and forcing the beneficial agents through an outlet port and into a body cavity at a predetermined rate. An anchoring mechanism may be associated with the housing for securing the housing inside the body cavity.

U.S. Pat. Nos. 5,167,626 and 5,170,801 to Casper at al., which are incorporated herein by reference, describe a capsule for releasing a substance at a defined location in the GI tract. The body of the capsule defines one or more apertures in the circumferential wall thereof, and a sleeve valve rotatably positioned therein has one or more corresponding apertures in the circumferential wall thereof. The sleeve valve comprises a coil and electrically connected heatable resistor which are operatively associated with an actuator member formed of a shape memory alloy responsive to heat and which will move from a non-heated first shape to a heated second shape. Actuator stop means are provided in the capsule body for being engaged by the actuator member during movement from the non-heated first shape to the heated second shape so that the actuator member movement serves to rotate the sleeve valve to an open position.

PCT Publication WO 01/45552 to Houzego et al., which is incorporated herein by reference, describes a closure member for a substance reservoir of a site-specific drug delivery capsule (SSDC). The SSDC includes a retainer that provides a non-linear force resisting opening of the closure member. The non-linear force is described as ensuring that the closure member unseals the reservoir only when an opening force exceeds a maximal value of the resisting force, thereby preventing premature or accidental emptying of the reservoir. The preferred means of providing the resistive force is a rolling, elastomeric o-ring that additionally seals the closure member into an aperture.

U.S. Pat. No. 6,344,027 to Goll, which is incorporated herein by reference, describes techniques for delivering and injecting fluid into heart tissue utilizing high pressure injection to increase injectate (fluid) retention in the heart tissue. A catheter is described which includes a shaft having an infusion lumen extending therethrough, wherein the proximal end of the shaft connected to a pressurized fluid source capable of generating a transient pressure of more than 1000 psi. The distal end of the shaft includes a nozzle having an injection port in fluid communication with the infusion lumen such that fluid from the pressurized fluid source may be delivered to the heart tissue at a sufficiently high exit velocity to partially penetrate the heart tissue.

U.S. Pat. No. 6,369,039 to Palasis et al., which is incorporated herein by reference, describes a method for site-specifically delivering a therapeutic agent to a target location within a body cavity, vasculature or tissue. The method comprises: providing a medical device having a substantially saturated solution of therapeutic agent associated therewith; introducing the medical device into the body cavity, vasculature or tissue; releasing a volume of the solution of therapeutic agent from the medical device at the target location at a pressure of from about 0 to about 5 atmospheres for a time of up to about 5 minutes; and withdrawing the medical device from the body cavity, vasculature or tissue. The patent also describes a system for delivering a therapeutic agent to a body cavity, vasculature or tissue, comprising a medical device having a substantially saturated solution of the therapeutic agent associated therewith.

U.S. Pat. No. 5,964,726 to Korenstein et al., which is incorporated herein by reference, describes techniques for introducing molecules and macromolecules into a membrane vesicle, a cell, or a tissue by (a) applying a train of low unipolar or alternating voltage pulses to molecules/macromolecules and cells, (b) increasing the concentration of the molecules/macromolecules at the surface of the cells, leading to an increased interaction of the molecules/macromolecules with the membrane of the cell while also causing electrophoretic movement of charged proteins and lipids in the cell membrane, and (c) causing the destabilization of the cell membrane whereby the molecules/macromolecules penetrate into the cytosol via an endocytic process and via diffusion through structural defects in the membrane lipid bilayer.

PCT Publication WO 02/098501 to Keisari et al., which is incorporated herein by reference, describes a method for treating tumor tissue, including applying to cells of the tumor tissue electrical field pulses having a strength, a repetition frequency, and a pulse width selected capable of inducing endocytosis-mediated cell death, thereby treating the tumor tissue.

U.S. Pat. No. 3,659,600 to Merrill, which is incorporated herein by reference, describes an implantable capsule activated by magnetic force to release a drug. U.S. Pat. Nos. 3,485,235 to Felson, U.S. Pat. No. 3,315,660 to Abella, U.S. Pat. No. 3,118,439 to Perrenoud, and U.S. Pat. No. 3,057,344 to Abella et al., which are incorporated herein by reference, describe capsules for insertion into the GI tract for treatment and/or diagnostic purposes.

An article by Lambert et al., entitled, “Autonomous telemetric capsule to explore the small bowel,” Med Biol Eng Comput 29(2):191-6 (1991), which is incorporated herein by reference, describes an intestinal telemetric capsule developed to study the small bowel in man. It consists of a cylinder (11 mm in diameter and 39 mm in length) containing a location detector, a radiotransmitter, a lithium battery and an interchangeable tip. After having been swallowed by the patient, the capsule passes through the whole gut and is recovered in the stool. During the transit through the small bowel, the information provided by the radiotransmitter allows continuous monitoring of the distance covered from the pylorus, as well as the direction and the velocity of progression. Moreover, according to the type of interchangeable tip, it is possible, by remote control, to sample 0.5 ml of intraluminal fluid for subsequent analysis or to release 1 ml of any liquid substance in a precisely-determined place for pharmacological studies.

There is a significant potential for novel oral drug delivery systems and methods, able to deliver drugs that are currently available only by injections, or that have poor and erratic bioavailability.

### SUMMARY OF THE INVENTION

In some embodiments of the present invention, an ingestible active drug-delivery system comprises electrical and/or mechanical means to enhance the absorption of a drug provided to the gastrointestinal (GI) tract. For some applications, such means includes a device for performing electrotransport of the drug, in order to actively deliver the drug through the wall of the GI tract. Alternatively or additionally, such means includes a mechanical driving mechanism that actively drives the drug through the wall of the GI tract. Typically, the drug-delivery system comprises a pill-shaped and -sized capsule that comprises the delivery means, and holds the drug until it is released to the GI tract.

Typically, the active driving of the drug through the GI tract wall is accomplished by: (a) driving the drug through the wall by passage of the drug through tight junctions of the epithelial layer of the small intestine, and/or (b) driving the drug through the wall by penetrating the epithelial cells themselves. Typically, a therapeutically-significant portion of the drug is thereby passed into direct contact with the capillary supply of the GI tract, and therefrom into the systemic circulation. It is noted that this embodiment therefore typically allows entry into the bloodstream of drug molecules which would normally be largely excluded (e.g., due to size or chemical properties).

In some embodiments of the present invention, the drug-delivery system comprises a mechanism that is operative to be responsive to its environment, such as, for example, a pH-sensitive coating. The coating is typically configured, using techniques known in the art, to dissolve upon entering a small intestine of a patient. In accordance with other embodiments of the present invention, the environmentally-responsive mechanism comprises, for example, a sensor (such as an electronic sensor) a timer, a transmitter/receiver, or a camera.

In some embodiments of the present invention, the dissolving of the coating triggers activation of the driving means, which, in turn, actively drives drug through the wall of the GI tract wall. For some applications, the coating is configured to dissolve in a pH range typical of the small intestine.

In some embodiments of the present invention, the coating is applied at a first thickness over a first portion of the capsule, and at a second thickness over a second portion of the capsule. Alternatively or additionally, different types of coatings are applied to different portions of the capsule, e.g., in order to provide for the respective portions of the capsule to be exposed to the small intestine at different times.

In some embodiments of the present invention, the driving mechanism comprises a gas generator and a movable member, such as a membrane. The membrane moves within the capsule in response to the generation of gas by the generator. In other configurations, the movable member comprises a piston. In yet other configurations, a movable member is not provided, but instead the gas generator acts directly on the drug.

For some applications, the dissolving of the coating activates the gas generator to release a gas that deflects the membrane. This deflection, in turn, applies pressure to the drug, driving it out of the capsule (typically through an orifice thereof), through the epithelial layer of the GI tract, and into contact with the capillary circulation of the GI tract.

For some applications, the gas generator comprises a power source, such as a battery, having positive and negative poles thereof coupled to respective electrodes. One of the electrodes is typically in contact with a liquid, such as a saline solution contained within the capsule. The solution, in turn, is typically in contact with or otherwise mechanically coupled to the membrane. Another one of the electrodes is typically mounted to an external surface of the capsule, within the coating. In addition, the capsule comprises an electrode having a first electrode contact electrically coupled to the solution, and a second electrode contact mounted to the outer surface of the capsule.

In these embodiments, the coating typically has very low electrical conductivity, and can be generally considered to act as an electrical insulator. Thus, when the coating is still present (e.g., before ingestion, and while the capsule is in the patient's stomach), the current drain from the battery is minimal or essentially zero. After entry of the capsule into the small intestine and upon the dissolving of the coating, the externally-mounted electrode and the externally-mounted electrode contact are electrically coupled via the ion-rich fluids naturally present in the small intestine. A current is thereby able to flow, powered by the battery, from the electrode in contact with the saline solution, via the solution, to the electrode contact electrically coupled to the solution. The flow of the current through the solution is associated with electrolysis of the water, and generates a gas. The gas generated by this process deflects the membrane and forces the drug out of the orifice, as described hereinabove.

In some embodiments of the present invention, the gas generator comprises a hydrophilic membrane and a substance typically adjacent to the hydrophilic membrane. The hydrophilic membrane is typically embedded in or otherwise coupled to the outer surface of the capsule. The substance is typically disposed within the capsule, and has the characteristic of rapidly releasing gas upon contact with the fluid of the GI tract. The hydrophilic membrane is protected from the fluid of the GI tract by the coating until the capsule arrives at a suitable region of the GI tract, such as a portion of the small intestine having a particular pH. At this point, the coating dissolves, and the hydrophilic membrane allows passage of the GI tract fluid into the capsule, where it contacts the substance. Gas is released rapidly in response to the reaction of the GI tract fluid with the substance. In turn, the drug is ejected at high pressure and velocity through the orifice and through the wall of the small intestine.

In some embodiments of the present invention, the gas generator comprises a hydrophilic membrane, as described hereinabove, and two electrodes typically but not necessarily embedded in the casing of the capsule. The electrodes typically comprise different metals. A conductor electrically couples the electrodes to one another. Typically, the conductor and the electrodes are encased within an insulator, and, in combination, constitute a galvanic cell. After the coating dissolves in response to the pH of the small intestine, fluid from the GI tract enters the capsule via the hydrophilic membrane. The fluid, once inside the capsule, provides (a) a low resistance pathway for current flow between the electrodes, and, in parallel, (b) the water source for electrolysis and corresponding rapid production of gas. The released gas, as described hereinabove, drives the drug out of the orifice and through the intestinal wall.

In some embodiments of the present invention, the gas generator comprises a hydrophilic membrane and one or more gas-releasing elements. The gas-releasing elements react with the acidic GI tract fluid passing through the hydrophilic membrane after the dissolving of the coating. This reaction rapidly releases gas, and drives the drug through the orifice of the capsule and through the epithelial layer of the small intestine.

In some embodiments of the present invention, the driving mechanism comprises a piston and a piston driver. For some applications, the piston driver comprises a mechanical spring. For other applications, the piston driver comprises a source of compressed air. In accordance with these embodiments, the capsule is typically stored with the piston driver in the tense state. The piston driver is prevented from releasing its energy by a portion of the coating that is disposed in a position within the capsule that inhibits motion of the piston. After ingestion of the capsule and the dissolving of the coating in the small intestine, the portion of the coating is exposed to the acidic environment of the small intestine, and dissolves as well, thereby freeing the piston. After the piston is released, the piston driver drives the piston to force the drug through the orifice and through the wall of the small intestine.

In some embodiments of the present invention, the capsule comprises a drug stored in powder form. A hydrophilic membrane, in addition to any uses it may have in activating the driving mechanism as described hereinabove, allows fluid from the GI tract to mix with the drug. Typically, the capsule is configured to facilitate this mixing prior to activation of the driving mechanism. In an embodiment, this pre-mixing of the drug with the GI tract fluid is brought about by setting a first thickness of the coating to be lower in a region surrounding the hydrophilic membrane than a second thickness of the coating in a region surrounding the driving mechanism. In this manner, the pH-sensitive coating over the hydrophilic membrane essentially completely dissolves, allowing the GI tract fluid to enter the capsule and mix with the drug. During this process, the portion of the coating over the driving mechanism is not yet sufficiently small to cause the activation of the driving mechanism. Subsequently, the portion of the coating over the driving mechanism also dissolves, causing the activation of the driving mechanism. This activation causes the (now substantially liquefied) drug to be (a) driven out of the orifice and (b) driven through the wall of the small intestine by the mechanical force applied thereto by the driving mechanism.

In some embodiments of the present invention, the capsule comprises a hollow needle located adjacent to the orifice and in communication with the drug. In the resting phase, one or more elastic elements hold the hollow needle generally within the capsule, such that the sharp tip of the needle does not extend past the coating, and, typically, does not extend past the outer surface of the capsule. As appropriate, the elastic elements may comprise springs, spring-like mechanical elements, or compressed air.

Upon activation of the driving mechanism, a substantial force is generated by the drug upon the needle. This force surpasses the force generated by the elastic elements, and thrusts the hollow needle out of the body of the capsule and through the wall of the small intestine. While the pressure within the capsule is still high, the drug passes through the channel in the hollow needle, past the endothelial layer of the small intestine, and into contact with the underlying capillary bed. When the high pressure subsequently declines, the force provided by the elastic elements surpasses that generated by the driving mechanism, and the hollow needle retracts within the body of the capsule.

In some embodiments of the present invention, the functionality for activating the driving mechanism, described hereinabove as being provided by a coating, is supplemented or replaced by other activating functionalities. For some applications, the capsule comprises a bio-sensor that detects a biological or physiological parameter, and activates the driving mechanism responsive thereto. As appropriate, the bio-sensor may comprise one or more of the following: an enzymatic sensor, a temperature sensor, a pH sensor, or a timer (the timer typically comprising chemicals that react in a known manner to activate the driving mechanism at a predetermined time following an event such as the patient squeezing the capsule or the patient ingesting the capsule). Alternatively or additionally, the capsule comprises a camera, which records an image of the GI tract for on-board analysis and, if appropriate, activation of the driving mechanism in response to the image.

For some applications, the capsule comprises a transmit/receive unit, adapted to transmit a signal responsive to an image recorded by the camera and/or responsive to a reading by the bio-sensor. The transmitted data are typically analyzed in real-time, and a decision is made (e.g., by a physician or by a computer external to the patient) whether and when to administer drug.

According to one aspect of the present invention there is provided an electrically-assisted drug-delivery system, comprising:

a biologically inert and biologically compatible device, comprising:

a power supply;

a control component, in power communication with the power supply; and

at least one apparatus for electrically assisted drug transport, the apparatus being in signal communication with the control component and in power communication with the power supply; and

a drug, attached to the device.

According to an additional aspect of the present invention, the drug further includes pharmaceutically acceptable additives for absorption enhancement.

According to an additional aspect of the present invention, the drug further includes pharmaceutically acceptable additives for improved bioavailability.

According to an additional aspect of the present invention, the drug further includes pharmaceutically acceptable additives for controlled release.

According to an additional aspect of the present invention, the drug further includes pharmaceutically acceptable additives for pH-dependent controlled release.

According to an additional aspect of the present invention, the drug further includes pharmaceutically acceptable additives for time-dependent controlled release.

According to an additional aspect of the present invention, the at least one apparatus for electrically assisted drug transport comprises an apparatus for at least one electrotransport process.

According to an additional aspect of the present invention, the apparatus for electrotransport is further operative to enhance peristalsis, by electrostimulation.

According to an additional aspect of the present invention, the apparatus for electrically assisted drug transport, comprises an apparatus for at least two electrotransport processes.

According to another aspect of the present invention, the at least one apparatus for electrically assisted drug transport comprises an apparatus for sonophoresis.

According to another aspect of the present invention, the at least one apparatus for electrically assisted drug transport comprises an apparatus for at least one ablation process.

According to an additional aspect of the present invention, the at least one apparatus for electrically assisted drug transport comprises an apparatus for at least two processes, selected from the group consisting of electrotransport, sonophoresis, and ablation.

According to an additional aspect of the present invention, the device includes at least one self-expansible portion, for making good contact with the gastrointestinal (GI) walls.

According to an additional aspect of the present invention, the power supply is a galvanic cell, which uses GI fluids as an electrolyte.

According to an additional aspect of the present invention, the device further defines a drug-dispensing cavity.

According to an additional aspect of the present invention, the drug-dispensing cavity is adapted for controlled release.

According to an additional aspect of the present invention, the drug-dispensing cavity is adapted for pH-dependent controlled release.

According to an additional aspect of the present invention, the drug-dispensing cavity is self-expansible, to make better contact with the GI walls.

According to an additional aspect of the present invention, the device includes a pH sensor.

According to an additional aspect of the present invention, the device includes a telemetry system for communicating with an extracorporeal station.

According to an additional aspect of the present invention, the device is ingestible.

According to an alternative aspect of the present invention, the device is attached to a catheter.

According to an additional aspect of the present invention, the device includes an imaging apparatus.

According to another aspect of the present invention there is provided a method of oral drug delivery, comprising:

orally inserting a drug into the GI tract; and

inducing transport through the GI walls, by a method selected from the group consisting of at least one electrotransport process, sonophoresis, at least one ablation process, and a combination thereof.

There is therefore provided, in accordance with an embodiment of the present invention, apparatus for drug administration, including:

an ingestible capsule, which includes:

a drug, stored by the capsule;

an environmentally-sensitive mechanism, adapted to change a state thereof responsive to a disposition of the capsule within a gastrointestinal tract of a subject; and

a driving mechanism, which, in response to a change of state of the environmentally-sensitive mechanism, is adapted to drive the drug directly through an endothelial layer of the gastrointestinal tract.

In an embodiment, the drug is stored in the capsule in liquid form.

In an embodiment, the environmentally-sensitive mechanism is adapted to undergo the change of state when the capsule is in a small intestine of the subject.

In an embodiment, the environmentally-sensitive mechanism is adapted to undergo the change of state when the capsule is in a large intestine of the subject.

In an embodiment, the environmentally-sensitive mechanism is adapted to undergo the change of state when the capsule is in a stomach of the subject.

In an embodiment, the environmentally-sensitive mechanism is essentially entirely biodegradable.

In an embodiment, the driving mechanism is essentially entirely biodegradable.

In an embodiment:

the environmentally-sensitive mechanism includes a sensor adapted to sense an indication of a distance traveled by the capsule in the gastrointestinal tract, and

the environmentally-sensitive mechanism is adapted to undergo the change of state responsive to the distance.

In an embodiment, the sensor includes an inertial sensor.

In an embodiment, at least 80% of the mass of the capsule is biodegradable.

In an embodiment, at least 95% of the mass of the capsule is biodegradable.

In an embodiment, essentially the entire capsule is biodegradable.

In an embodiment, the capsule includes a self-expansible portion, which is adapted to expand responsive to the change of state of the environmentally-sensitive mechanism.

In an embodiment, a characteristic diameter of the self-expansible portion is adapted to increase by at least 100%, responsive to the change of state of the environmentally-sensitive mechanism.

In an embodiment, the self-expansible portion is adapted to expand responsive to expansion of a gas within the self-expansible portion.

In an embodiment, the self-expansible portion is adapted to expand responsive to an inflow of fluid from the gastrointestinal tract.

In an embodiment:

a characteristic diameter of the self-expansible portion immediately prior to expanding is smaller than a characteristic diameter of a portion of the gastrointestinal tract containing the capsule, and

a characteristic diameter of the self-expansible portion following expanding is at least as large as a characteristic diameter of the portion of the gastrointestinal tract containing the capsule.

In an embodiment:

the capsule includes an electrode on an outer surface of the self-expansible portion, and

the driving mechanism is adapted to drive current through the electrode when the self-expansible portion is in an expanded state thereof.

In an embodiment:

the self-expansible portion includes a first self-expansible portion, at a first end of the capsule,

the capsule includes a second self-expansible portion, at a second end of the capsule, and

the capsule includes an electrode on an outer surface of the second self-expansible portion.

In an embodiment, the capsule includes a third self-expansible portion, disposed between the first and second self-expansible portions.

In an embodiment, the capsule includes an electrode on an outer surface of the third self-expansible portion.

In an embodiment, the capsule contains no electrodes on an outer surface of the third self-expansible portion.

In an embodiment, the environmentally-sensitive mechanism includes a coating on a surface of the capsule.

In an embodiment, the coating includes a pH-sensitive coating.

In an embodiment, the pH-sensitive coating is sensitive to a pH that is characteristic of a small intestine.

In an embodiment:

the coating is adapted to cover a portion of the driving mechanism, prior to the change of state, in a manner that substantially prevents contact of the driving mechanism with a first fluid of the gastrointestinal tract, and

the coating is adapted to uncover the portion of the driving mechanism in response to the coating contacting a second fluid of the gastrointestinal tract.

In an embodiment, the driving mechanism is adapted to drive the drug directly through the endothelial layer of the gastrointestinal tract responsive to uncovering of the portion of the driving mechanism.

In an embodiment, the environmentally-sensitive mechanism includes a timer, adapted to change the state of the environmentally-sensitive mechanism responsive to a duration of the capsule in the gastrointestinal tract.

In an embodiment, the timer includes an electronic timer.

In an embodiment, the timer includes a chemical timer, adapted to change the state of the environmentally-sensitive mechanism responsive to a chemical reaction.

In an embodiment, the environmentally-sensitive mechanism includes a camera, adapted to image the gastrointestinal tract, and the driving mechanism is adapted to drive the drug through the endothelial layer in response to an image acquired by the camera.

In an embodiment, the capsule includes a control component, adapted to interpret the acquired image and activate the driving mechanism responsive thereto.

In an embodiment, the capsule includes a transmit/receive unit, adapted to transmit data responsive to the acquired image, to receive an instruction responsive to the transmission, and to activate the driving mechanism responsive to the instruction.

In an embodiment, the environmentally-sensitive mechanism includes a sensor, adapted to sense a characteristic of the gastrointestinal tract, and the driving mechanism is adapted to drive the drug through the endothelial layer in response to the sensed characteristic.

In an embodiment, the capsule includes a control component, adapted to interpret the sensed characteristic and activate the driving mechanism responsive thereto.

In an embodiment, the capsule includes a transmit/receive unit, adapted to transmit data responsive to the sensed characteristic, to receive an instruction responsive to the transmission, and to activate the driving mechanism responsive to the instruction.

In an embodiment, the sensor includes an enzymatic sensor.

In an embodiment, the sensor includes an optical sensor.

In an embodiment, the sensor includes a thermal sensor.

In an embodiment, the sensor includes a pH sensor. In an embodiment, the pH sensor is adapted to detect a pH between about 4.7 and about 6.5. In an embodiment, the pH sensor is adapted to detect a pH between about 1.2 and about 3.5. In an embodiment, the pH sensor is adapted to detect a pH between about 4.6 and about 6.0. In an embodiment, the pH sensor is adapted to detect a pH between about 7.5 and about 8.0.

In an embodiment, the sensor includes a sensor adapted to detect a pathological condition of the gastrointestinal tract. In an embodiment, the sensor includes a sensor adapted to detect bleeding in the gastrointestinal tract. In an embodiment, the sensor includes a sensor adapted to detect inflammation in the gastrointestinal tract.

In an embodiment:

the capsule includes a needle including a sharp tip thereof, and

the tip of the needle is adapted to contact the endothelial layer of the gastrointestinal tract in response to the change of state of the environmentally-sensitive mechanism.

In an embodiment, the needle is hollow.

In an embodiment, the needle is not hollow.

In an embodiment:

the capsule includes an elastic element, adapted to maintain the sharp tip of the needle at an original position that is substantially within the capsule, prior to the change of state,

in response to an action of the driving mechanism, the elastic element is adapted to change shape in a manner that permits the sharp tip of the needle to contact the endothelial layer of the gastrointestinal tract, and

at a time after initiation of the driving of the drug through the endothelial layer, the elastic element is adapted to cause the sharp tip of the needle to withdraw to the original position.

In an embodiment, the driving mechanism is adapted to drive the needle to puncture the endothelial layer of the gastrointestinal tract at a puncture site, in response to the change of state of the environmentally-sensitive mechanism.

In an embodiment, the driving mechanism is adapted to drive the drug through the puncture site.

In an embodiment, the drug is stored in the capsule in powder form.

In an embodiment, the capsule is adapted to mix the drug in powder form with a fluid, in response to the change of state of the environmentally-sensitive mechanism.

In an embodiment:

the fluid includes fluid of the gastrointestinal tract, and

the capsule is adapted to mix the drug in powder form with the gastrointestinal tract fluid, in response to the change of state of the environmentally-sensitive mechanism.

In an embodiment:

the fluid includes fluid stored within the capsule, separately from the drug in powder form, and

the capsule is adapted to mix the drug in powder form with the fluid stored within the capsule, in response to the change of state of the environmentally-sensitive mechanism.

In an embodiment:

the driving mechanism includes a control component, a first electrode, a second electrode, and a third electrode,

the control component is adapted to drive an iontophoretic current between the first and second electrodes, and

the control component is adapted to drive an electropulsation current through the third electrode.

In an embodiment:

the driving mechanism includes a control component, a first electrode, and a second electrode, and

the control component is adapted to drive a current between the first and second electrodes in response to the change of state of the environmentally-sensitive mechanism.

In an embodiment, the environmentally-sensitive mechanism includes a coating on a surface of the capsule.

In an embodiment, the driving mechanism includes the first and second electrodes and no other electrodes.

In an embodiment, the driving mechanism includes more than three electrodes.

In an embodiment, the control component is adapted to configure the current to ablate at least a portion of the endothelial layer of the gastrointestinal tract.

In an embodiment, the control component includes a battery. In an embodiment, the battery is biodegradable. In an embodiment, the battery includes zinc and manganese dioxide.

In an embodiment, the driving mechanism includes a third electrode, and the control component is adapted to drive a current between the first and third electrodes in response to the change of state of the environmentally-sensitive mechanism.

In an embodiment, the first electrode is physically disposed on the capsule between the second electrode and the third electrode.

In an embodiment, the control component is adapted to configure the current driven between the first and second electrodes to be substantially identical to the current driven between the first and third electrodes.

In an embodiment:

the control component is adapted to configure the current driven between the first and second electrodes to consist essentially of an iontophoretic current, and

the control component is adapted to configure the current driven between the first and third electrodes to consist essentially of an electropulsation current.

In an embodiment, the control component is adapted to drive the current between the first and second electrodes at a level sufficient to iontophoretically drive the drug through the endothelial layer of the gastrointestinal tract.

In an embodiment, the control component is adapted to configure a voltage drop between the first and second electrodes to be less than about 3 volts.

In an embodiment, the control component is adapted to configure the current to be substantially DC.

In an embodiment, the control component is adapted to configure the current to have a characteristic frequency less than about 50 Hz. In an embodiment, the control component is adapted to configure the current to have a characteristic frequency less than about 5 Hz.

In an embodiment, the control component is adapted to configure the current to have an amplitude less than about 5 mA. In an embodiment, the control component is adapted to configure the current to have an amplitude greater than about 0.5 mA.

In an embodiment, the control component is adapted to configure the current to increase conduction of the drug through tight junctions of the endothelial layer of the gastrointestinal tract by means of electropulsation.

In an embodiment, the control component is adapted to configure a voltage drop between the first and second electrodes to be between about 3 and about 12 volts.

In an embodiment, the control component is adapted to configure a voltage drop between the first and second electrodes to be between about 12 and about 50 volts.

In an embodiment, the control component is adapted to configure the current to have a characteristic frequency less than about 300 Hz. In an embodiment, the control component is adapted to configure the current to have a characteristic frequency less than about 100 Hz. In an embodiment, the control component is adapted to configure the current to have a characteristic frequency greater than about 1 Hz. In an embodiment, the control component is adapted to configure the current to have a characteristic frequency greater than about 10 Hz. In an embodiment, the control component is adapted to configure the current to have a characteristic frequency less than about 20 Hz. In an embodiment, the control component is adapted to configure the current to have a characteristic frequency greater than about 10 Hz.

In an embodiment, the control component is adapted to configure the current to: (a) be at a level sufficient to iontophoretically drive the drug through the endothelial layer of the gastrointestinal tract, and (b) increase conduction of the drug through tight junctions of the endothelial layer of the gastrointestinal tract by means of electropulsation.

In an embodiment:

the current includes an iontophoretic current and an electropulsation current,

the control component is adapted to drive the iontophoretic current between the first and second electrodes, and

the control component is adapted to drive the electropulsation current between the first and second electrodes.

In an embodiment, the control component is adapted to configure the current to have a high-frequency component and a low-frequency component. In an embodiment, the control component is adapted to configure the high-frequency component and the low-frequency component to have frequencies that are respectively greater than and less than 5 Hz.

In an embodiment, the control component is adapted to drive the high-frequency component and the low-frequency component at the same time.

In an embodiment, the control component is adapted to drive the high-frequency component prior to driving the low-frequency component. In an embodiment, the control component is adapted to initiate driving the high-frequency component at least 30 seconds prior to driving the low-frequency component.

In an embodiment, the driving mechanism includes a piston and a piston driver, and the piston driver is adapted to drive the piston to drive the drug from the capsule.

In an embodiment, the piston driver includes a compressed gas that is adapted to expand in response to the change of state of the environmentally-sensitive mechanism.

In an embodiment, the piston driver includes a spring-like mechanical element.

In an embodiment, the driving mechanism includes a gas generator, which, in response to the change of state of the environmentally-sensitive mechanism, is adapted to generate a gas which on expansion thereof performs work on the drug in a manner that drives the drug from the capsule and directly through the endothelial layer of the gastrointestinal tract.

In an embodiment, the gas generator is adapted to generate, within about 1 minute, a pressure change of at least 0.2 atmosphere within the capsule, in response to the change of state of the environmentally-sensitive mechanism.

In an embodiment, the gas generator is adapted to generate, within about 20 minutes, a pressure change of at least 0.2 atmosphere within the capsule, in response to the change of state of the environmentally-sensitive mechanism.

In an embodiment:

the capsule includes a flexible membrane between the gas generator and the drug,

the membrane is adapted to be deflected in response to the generation of the gas, and

the membrane, in response to being deflected, is adapted to drive the drug through the endothelial layer of the gastrointestinal tract.

In an embodiment, the gas generator is in a common compartment with the drug, and the gas generated by the gas generator, in direct contact with the drug, drives the drug from the capsule and directly through the endothelial layer of the gastrointestinal tract.

In an embodiment, the gas generator is adapted to generate a pressure change of at least about 0.1 atmosphere within the capsule, in response to the change of state of the environmentally-sensitive mechanism.

In an embodiment, the gas generator is adapted to configure the pressure change to be less than about 5 atmospheres, in response to the change of state of the environmentally-sensitive mechanism.

In an embodiment, the gas generator is adapted to configure the pressure change to be between about 0.5 and 3 atmospheres, in response to the change of state of the environmentally-sensitive mechanism.

In an embodiment, the gas generator is adapted to configure the pressure change to occur during less than about 1 minute. In an embodiment, the gas generator is adapted to configure the pressure change to occur over a time period having a duration between about 1 and 10 minutes. In an embodiment, the gas generator is adapted to configure the pressure change to occur over a time period having a duration between about 10 and 120 minutes.

In an embodiment, the gas generator is adapted to facilitate entry into the capsule of fluid of the gastrointestinal tract in response to the change of state of the environmentally-sensitive mechanism, and to generate the gas responsive to the entry of the gastrointestinal tract fluid into the capsule.

In an embodiment:

the gas generator includes a substance, and

the gas generator is adapted to generate the gas by causing contact of the gastrointestinal tract fluid with the substance, in response to the change of state of the environmentally-sensitive mechanism.

In an embodiment, the substance includes a substance selected from the list consisting of elemental sodium and elemental calcium.

In an embodiment:

the gas generator includes a substance having a pH greater than 7, and

the gas generator is adapted to generate the gas by facilitating contact between the substance and fluid of the gastrointestinal tract, in response to the change of state of the environmentally-sensitive mechanism.

In an embodiment, the substance includes sodium bicarbonate.

In an embodiment, the gas generator includes a membrane proximate the substance, which is adapted to facilitate entry of the gastrointestinal tract fluid into the capsule, through the membrane, in response to the change of state of the environmentally-sensitive mechanism.

In an embodiment, the membrane includes a hydrophilic membrane.

In an embodiment, the membrane is integral to an outer surface of the capsule.

In an embodiment, the gas generator includes a galvanic cell.

In an embodiment, the galvanic cell includes a first electrode including zinc and a second electrode including manganese dioxide.

In an embodiment, the galvanic cell includes first and second galvanic cell electrodes, which are adapted to pass current through fluid of the gastrointestinal tract at a level sufficient to electrolyze the fluid and generate the gas.

In an embodiment, the gas generator includes a membrane, which is adapted to facilitate entry of fluid of the gastrointestinal tract into the capsule, through the membrane, and into contact with the first and second galvanic cell electrodes, in response to the change of state of the environmentally-sensitive mechanism.

In an embodiment:

an outer surface of the capsule is shaped so as to define an orifice having an edge, the edge of the orifice generally being in contact with a portion of the gastrointestinal tract at a time after the environmentally-sensitive mechanism changes state, and

the gas generator and the drug are disposed within the capsule in such a manner that the generation of the gas drives the drug through the orifice and, therefrom, through the portion of the gastrointestinal tract.

In an embodiment, the capsule includes a seal, which blocks the orifice prior to the change of state of the environmentally-sensitive mechanism, and which is adapted to be removed from the orifice in response to the generation of the gas by the gas generator.

In an embodiment, the seal includes a plug, adapted to:

be disposed within the orifice prior to the change of state of the environmentally-sensitive mechanism,

resist ejection from the orifice during an initial rise in pressure within the capsule that occurs in response to the generation of the gas by the gas generator, and

be ejected from the orifice when the pressure within the capsule surpasses a threshold pressure.

In an embodiment, the capsule is shaped such that a characteristic diameter of the orifice is between about 20 and about 400 microns. In an embodiment, the capsule is shaped such that the characteristic diameter of the orifice is between about 20 and about 50 microns. In an embodiment, the capsule is shaped such that the characteristic diameter of the orifice is between about 50 and about 300 microns.

In an embodiment, the gas generator includes an electrical power source, adapted to drive current through a fluid in a manner that causes the generation of the gas by electrolysis of the fluid.

In an embodiment:

the power source includes first and second poles,

the gas generator includes the fluid,

the first pole of the power source is directly electrically coupled to the fluid,

the gas generator includes a coupling electrode, electrically coupled to the second pole of the power source,

the gas generator includes a second electrode, electrically coupled via the fluid to the first pole of the power source, and substantially electrically isolated from the coupling electrode prior to the change of state of the environmentally-sensitive mechanism, and

the environmentally-sensitive mechanism is adapted, in response to the change of state, to establish electrical contact between the coupling electrode and the second electrode.

In an embodiment, the fluid includes fluid of the gastrointestinal tract, and the gas generator is adapted, in response to the change of state of the environmentally-sensitive mechanism, to drive the current through the fluid of the gastrointestinal tract.

There is further provided, in accordance with an embodiment of the present invention, apparatus for administration of a drug, including:

an ingestible capsule adapted to store the drug, the capsule including:

an environmentally-sensitive mechanism, adapted to change a state thereof responsive to a disposition of the capsule within a gastrointestinal tract of a subject; and

a driving mechanism, which, in response to a change of state of the environmentally-sensitive mechanism, is adapted to drive the drug directly through an endothelial layer of the gastrointestinal tract.

There is further provided, in accordance with an embodiment of the present invention, apparatus for administration of a drug, including:

an ingestible environmentally-sensitive mechanism, adapted to change a state thereof responsive to a disposition thereof within a gastrointestinal tract of a subject; and

a driving mechanism, which, in response to a change of state of the environmentally-sensitive mechanism, is adapted to drive the drug directly through an endothelial layer of the gastrointestinal tract.

There is yet further provided, in accordance with an embodiment of the present invention, apparatus, including:

a capsule adapted to travel through a gastrointestinal tract of a subject, the capsule including:

first and second electrodes; and

a control component, adapted to drive, at each of a plurality of sites longitudinally distributed along the gastrointestinal tract, an iontophoretic current that travels from the first electrode, through an endothelial layer of the gastrointestinal tract, and to the second electrode.

In an embodiment, the control component is adapted to drive the iontophoretic current while the capsule is in motion.

In an embodiment, the control component is adapted to configure a voltage drop between the first and second electrodes to be less than about 3 volts, and to configure a characteristic frequency of the iontophoretic current to be less than about 5 Hz.

In an embodiment, the capsule includes a self-expansible portion, and the first electrode is disposed on an outer surface of the self-expansible portion.

In an embodiment, the capsule includes a second self-expansible portion, and the second electrode is disposed on an outer surface of the second self-expansible portion.

In an embodiment, the capsule includes a coating on an outer surface thereof, and the control component is adapted to initiate driving the iontophoretic current in response to a change of state of the coating.

There is still further provided, in accordance with an embodiment of the present invention, apparatus, including:

a capsule adapted to travel through a gastrointestinal tract of a subject, the capsule including:

first and second electrodes; and

a control component, adapted to drive, at each of a plurality of sites longitudinally distributed along the gastrointestinal tract, an electropulsation current that travels from the first electrode, through an endothelial layer of the gastrointestinal tract, and to the second electrode.

In an embodiment, the control component is adapted to drive the electropulsation current while the capsule is in motion.

In an embodiment, the control component is adapted to configure a voltage drop between the first and second electrodes to be greater than about 3 volts, and to configure a characteristic frequency of the electropulsation current to be between about 1 and 30 Hz.

In an embodiment, the capsule includes a self-expansible portion, and the first electrode is disposed on an outer surface of the self-expansible portion.

In an embodiment, the capsule includes a second self-expansible portion, and the second electrode is disposed on an outer surface of the second self-expansible portion.

In an embodiment, the capsule includes a coating on an outer surface thereof, and the control component is adapted to initiate driving the electropulsation current in response to a change of state of the coating.

There is also provided, in accordance with an embodiment of the present invention, apparatus, including:

a capsule adapted to travel through a gastrointestinal tract of a subject, the capsule including:

first and second electrodes;

a coating on an outer surface of the capsule; and

a control component, adapted to drive an iontophoretic current that travels from the first electrode, through an endothelial layer of the gastrointestinal tract, and to the second electrode, in response to a change of state of the coating.

In an embodiment, the capsule includes first and second self-expansible portions, at respective ends of the capsule, and the first and second electrodes are disposed on respective outer surfaces of the first and second self-expansible portions.

There is additionally provided, in accordance with an embodiment of the present invention, a method for administration of a drug, including:

administering to a subject an ingestible capsule that includes a drug;

detecting a disposition of the capsule within a gastrointestinal tract of the subject; and

in response to detecting the disposition, driving the drug directly through an endothelial layer of the gastrointestinal tract.

In an embodiment, driving the drug includes iontophoretically driving the drug.

In an embodiment, driving the drug includes applying an electropulsation current configured to facilitate the driving of the drug.

In an embodiment, driving the drug includes expanding a portion of the capsule in response to detecting the disposition.

In an embodiment, detecting the disposition includes causing an interaction between a coating on an outer surface of the capsule and fluid of the gastrointestinal tract.

Embodiments of the present invention successfully address the shortcomings of the presently-known configurations by providing a typically ingestible, electrically or mechanically assisted, drug-delivery system, which acts as a medication carrier, and which utilizes electrically or mechanically induced means to enhance the absorption of the medication through the GI walls.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of the intestinal wall;

FIG. 2 is a schematic illustration of a device for electrically-assisted drug delivery, in accordance with some embodiments of the present invention;

FIGS. 3A and 3B are schematic illustrations of ingestible, electrically-assisted drug-delivery systems, in accordance with embodiments of the present invention;

FIG. 4 is a schematic illustration of an ingestible, electrically-assisted drug-delivery system, having a plurality of electrodes, in accordance with an embodiment of the present invention;

FIG. 5 is a schematic illustration of another ingestible, electrically-assisted drug-delivery system, having a plurality of electrodes, in accordance with an embodiment of the present invention;

FIGS. 6A and 6B are schematic illustrations of an ingestible, electrically-assisted drug-delivery system, having self-expansible portions, in accordance with embodiment of the present invention;

FIG. 7 is a schematic illustration of an ingestible, electrically-assisted drug-delivery system, having a plurality of electrodes, in accordance with an embodiment of the present invention;

FIG. 8 is a schematic illustration of an ingestible, electrically-assisted drug-delivery system, having a plurality of electrodes and self-expansible portions, in accordance with an embodiment of the present invention;

FIG. 9 is a schematic illustration of another ingestible, electrically-assisted drug-delivery system, having a plurality of electrodes and self-expansible portions, in accordance with an embodiment of the present invention;

FIG. 10 is a schematic illustration of an ingestible, electrically-assisted drug-delivery system, having a plurality of electrodes and self-expansible portions, when in the gastrointestinal tract, in accordance with an embodiment of the present invention;

FIGS. 11A-11D are schematic illustrations of an ingestible, electrically-assisted drug-delivery system, wherein the drug-dispensing cavities are formed as self-expansible portions, in accordance with embodiments of the present invention;

FIG. 12 is a schematic illustration of an ingestible, electrically-assisted drug-delivery system, having a drug cavity with a biodegradable cap, in accordance with an embodiment of the present invention;

FIG. 13 is a schematic illustration of an ingestible, electrically-assisted drug-delivery system, wherein the drug is pressed into an integrated tablet with the system, in accordance with an embodiment of the present invention;

FIGS. 14A and 14B are schematic illustrations of an ingestible, electrically-assisted drug-delivery system, adapted to form an osmosis pump in the gastrointestinal tract, in accordance with embodiments of the present invention;

FIG. 15 is a schematic illustration of an ingestible, electrically-assisted drug-delivery system, having a pH-dependent controlled drug release, in accordance with an embodiment of the present invention;

FIG. 16 is a schematic illustration of an ingestible, electrically-assisted drug-delivery system, having an electronically activated, pH-dependent controlled drug release, in accordance with an embodiment of the present invention;

FIG. 17 is a schematic illustration of an ingestible, electrically-assisted drug-delivery system, adapted for sonophoresis, in accordance with an embodiment of the present invention;

FIG. 18 is a schematic illustration of an ingestible, electrically-assisted drug-delivery system, adapted for ablation, in accordance with an embodiment of the present invention;

FIG. 19 is a schematic illustration of an ingestible, electrically-assisted drug-delivery system, adapted for telemetry communication, in accordance with an embodiment of the present invention;

FIG. 20 is a schematic illustration of an ingestible, electrically-assisted drug-delivery system, adapted to make a galvanic cell with the body, in accordance with an embodiment of the present invention;

FIGS. 21A and 21B are schematic illustrations of a drug-delivery system comprising a capsule, in accordance with embodiments of the present invention;

FIGS. 22A and 22B are schematic illustrations of a drug-delivery system comprising a gas generator, in accordance with an embodiment of the present invention;

FIGS. 23A and 23B are schematic illustrations of a drug-delivery system comprising a gas generator having a power source, in accordance with an embodiment of the present invention;

FIGS. 24A and 24B are schematic illustrations of a drug-delivery system comprising a gas generator having a hydrophilic membrane, in accordance with an embodiment of the present invention;

FIGS. 25A and 25B are schematic illustrations of another drug-delivery system comprising a gas generator having a hydrophilic membrane, in accordance with an embodiment of the present invention;

FIGS. 26A and 26B are schematic illustrations of yet another drug-delivery system comprising a gas generator having a hydrophilic membrane, in accordance with an embodiment of the present invention;

FIGS. 27A and 27B are schematic illustrations of a drug-delivery system comprising a piston, in accordance with an embodiment of the present invention;

FIGS. 28A, 28B, and 28C are schematic illustrations of a drug-delivery system comprising a drug stored in powder form, in accordance with an embodiment of the present invention;

FIGS. 29A and 29B are schematic illustrations of a drug-delivery system comprising a needle, in accordance with an embodiment of the present invention; and

FIG. 30 is a schematic illustration of a drug-delivery system, in accordance with an embodiment of the present invention.

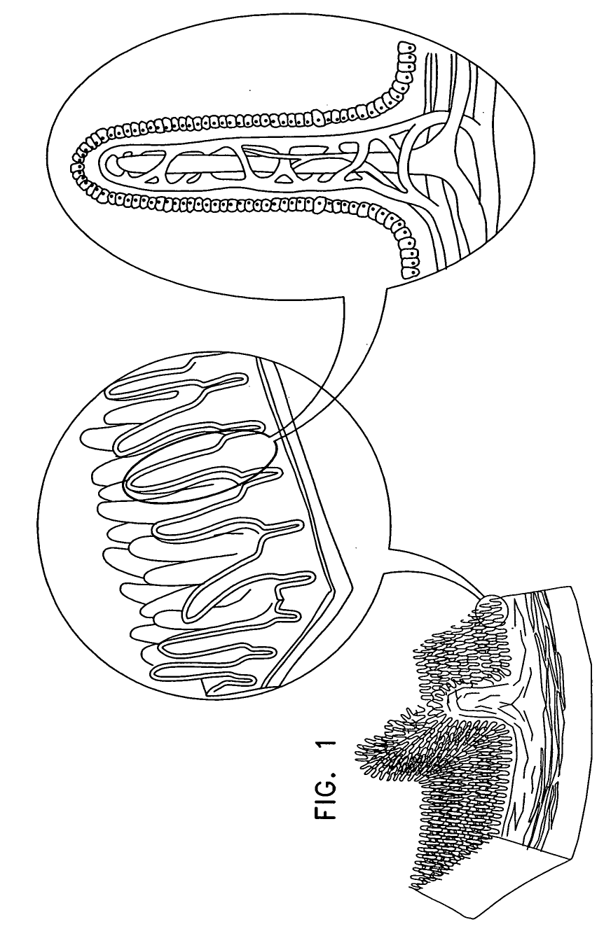

### DESCRIPTION OF EMBODIMENTS

Embodiments of the present invention comprise a typically ingestible, electrically- or mechanically-assisted, drug-delivery system. Specifically, these embodiments of the present invention act as a medication carrier, which utilizes electrically- or mechanically-induced means to enhance the absorption of the medication through the gastrointestinal (GI) tract walls.

The principles and operation of the typically ingestible, electrically- or mechanically-assisted, drug-delivery system, according to these embodiments of the present invention, may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

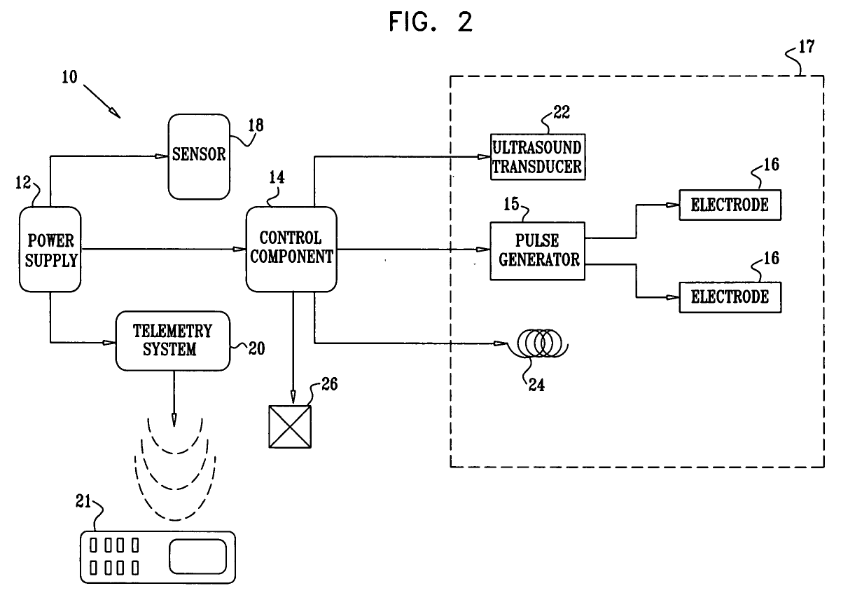

Referring now to the drawings, FIG. 2 is a schematic diagram of an electrically-assisted, drug-delivery device 10, in accordance with some embodiments of the present invention. Device 10 is biologically inert and biologically compatible, and is typically adapted for ingestion. Device 10 comprises a power supply 12, a control component 14 in power communication with power supply 12, and at least one apparatus 17 for electrically-assisted drug transport, which is in signal communication with control component 14 and in power communication with power supply 12. Control component 14 may be dedicated circuitry, a controller, or a microcomputer, as known in the art.

For some applications, apparatus 17 comprises a pulse generator 15 and at least two electrodes 16, designed for electrotransport. Alternatively, four or more electrodes 16 may be provided. Apparatus 17 may be designed, for example, as an electrotransport device, as described in any one, or a combination of, U.S. Pat. No. 5,674,196, to Donaldson et al., U.S. Pat. No. 5,961,482 to Chien et al., U.S. Pat. No. 5,983,131 to Weaver et al., U.S. Pat. No. 5,983,134 to Ostrow, and U.S. Pat. No. 6,477,410 to Henley et al., all of which are incorporated herein by reference.

Additionally or alternatively, apparatus 17 is designed for performing sonophoresis, or for performing a combination of sonophoresis and electrotransport, and comprises at least one ultrasound transducer 22. Apparatus 17 may be designed, for example, as a sonophoresis device, as described in any one, or a combination of, U.S. Pat. Nos. 6,002,961, 6,018,678, and 6,002,961 to Mitragotri et al., U.S. Pat. Nos. 6,190,315 and 6,041,253 to Kost et al., U.S. Pat. No. 5,947,921 to Johnson et al., and U.S. Pat. Nos. 6,491,657 and 6,234,990 to Rowe et al., all of which are incorporated herein by reference.

Additionally or alternatively, apparatus 17 is designed for performing ablation, or for performing a combination of ablation and electrotransport, ablation and sonophoresis, or ablation, electrotransport, and sonophoresis, and comprises at least one ablation apparatus 24. The ablation process may be, for example, any one of, or a combination of, laser ablation, cryogenic ablation, thermal ablation, microwave ablation, radiofrequency (RF) ablation, electrical ablation, and liquid jet ablation. Apparatus 17 may be designed, for example, as an ablation device, as described in any one, or a combination of, U.S. Pat. No. 6,471,696, to Berube et al. (which describes a microwave ablation catheter that may be used as a drug delivery device), U.S. Pat. No. 6,443,945 to Marchitto et al. (which describes a devices for pharmaceutical delivery using laser ablation), U.S. Pat. No. 4,869,248 to Narula (which describes a catheter for performing localized thermal ablation for drug administration), and U.S. Pat. Nos. 6,148,232 and 5,983,135 to Avrahami (which describe drug delivery systems using electrical ablation). All of these patents are incorporated herein by reference.

In accordance with some embodiments of the present invention, device 10 further comprises at least one sensor 18. Sensor 18 may be, for example, a physical sensor, such as a temperature sensor or a pressure sensor. Alternatively, sensor 18 may be a chemical sensor, such as a pH sensor or a drug-concentration sensor. Alternatively, sensor 18 may be a biological sensor, such as a glucose sensor or a bacterial-count sensor. For some applications, more than one sensor 18 is used. These may be of the same type or of different types.

In accordance with some embodiments of the present invention, device 10 further comprises a telemetry system 20, operative, for example, by RF, infrared radiation, or by ultrasound, for providing communication with an extracorporeal station 21, for example, a remote control. Alternatively or additionally, extracorporeal station 21 comprises a computer system. Alternatively or additionally, telemetry system 20 comprises a power transducer (such as a coil), as is known in the art, adapted to receive electromagnetic radiation transmitted by extracorporeal station 21, and to transduce the radiation into a current for powering the operation of drug-delivery device 10. As appropriate, the power transducer may replace power supply 12, or supplement its operation.

In accordance with some embodiments of the present invention, device 10 further comprises at least one electronic valve 26 for dispensing medication, for example, responsive to input from sensor 18.

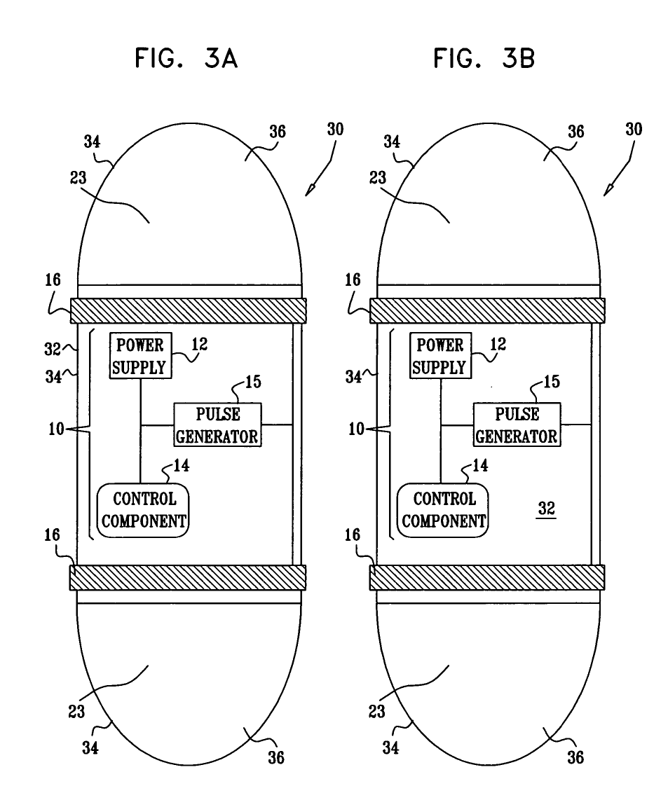

Reference is now made to FIGS. 3A and 3B, each of which illustrates an ingestible, electrically-assisted, drug-delivery system 30, in accordance with embodiments of the present invention. System 30 comprises device 10, enclosed within a biocompatible, biologically inert housing 32, formed for example, of stainless steel or silicone, or another biocompatible, inert material. Device 10 of the present embodiment typically comprises at least power supply 12, control component 14, pulse generator 15, and at least two electrostimulating electrodes 16, for providing electrotransport.

In the embodiment shown in FIG. 3A, housing 32 of device 10 defines an internal cavity in which components of device 10 are located. In the embodiment shown in FIG. 3B, housing 32 defines no cavity; rather, it is formed as a cast, for example of silicone, wherein components of device 10 are imbedded.

System 30 further comprises a drug 36, attached to device 10 and enclosed by a sheath 34, which encapsulates both device 10 and drug 36. Alternatively, sheath 34 encapsulates only drug 36. Drug 36 is held in drug-dispensing cavities 23, which typically are formed at two ends of system 30, or at one end. Sheath 34 typically comprises a biologically compatible, biologically inert polymeric material, such as cellulose acetate or ethyl cellulose, that allows diffusion of drug 36 to the GI tract. Alternatively, sheath 34 is formed of a mixture of water-soluble particles in a water-insoluble matrix, such as polyvinyl acetate, or acrylic acid copolymers, so that the water soluble particles dissolve in the GI tract, leaving micropores in matrix, and drug 36 diffuses through the micropores. Alternatively, sheath 34 is formed of biologically-degradable material, which degrades when in contact with water, or at a specific pH value, so as to release drug 36 to the GI tract, where drug 36 travels with device 10 until the drug is absorbed. For example, the biologically-degradable material may comprise hydroxypropylcellulose or glycerol behenate. As system 30 travels in the GI tract, electrodes 16 of device 10 provide for electrotransport, which enhances absorption across the intestinal epithelium.

In accordance with some embodiments of the present invention, the electrotransport may include any one of, or a combination of, iontophoresis, electroosmosis, and electrophoresis, which enhance diffusion processes through the epithelial cells, and, for some applications, additionally electroporation, which physically punctures or opens biological barriers, along the tight junctions of the epithelial cell boundaries, enabling passage of large molecules through the epithelium.

Appropriate electrostimulation parameters may include a DC voltage of up to 3 volts, or square pulses of up to 3 volts at a low frequency of 1-50 Hz. These parameters are typically appropriate for iontophoresis. Alternatively, the parameters may include an AC voltage of between 3 and 50 Volts, at a frequency of between 1 and 300 Hz. These parameters are typically appropriate for electroporation. Additionally, for some applications, the DC or low-frequency square-pulse voltage and the AC voltage are superimposed, in order to perform a combination of two or more electrotransport processes.

It will be appreciated that pulses of other shapes and (or) duty cycles may similarly be used. Furthermore, the aforementioned parameters are provided as examples; in accordance with embodiments of the present invention, other parameters, which may be higher or lower, may be used.

It will be appreciated that, in general, electrotransport parameters appropriate for the transport of drugs across the epithelial cells of the GI tract are lower than parameters appropriate for transdermal drug transport, as the GI tract lacks the stratum corneum barrier found in the skin.

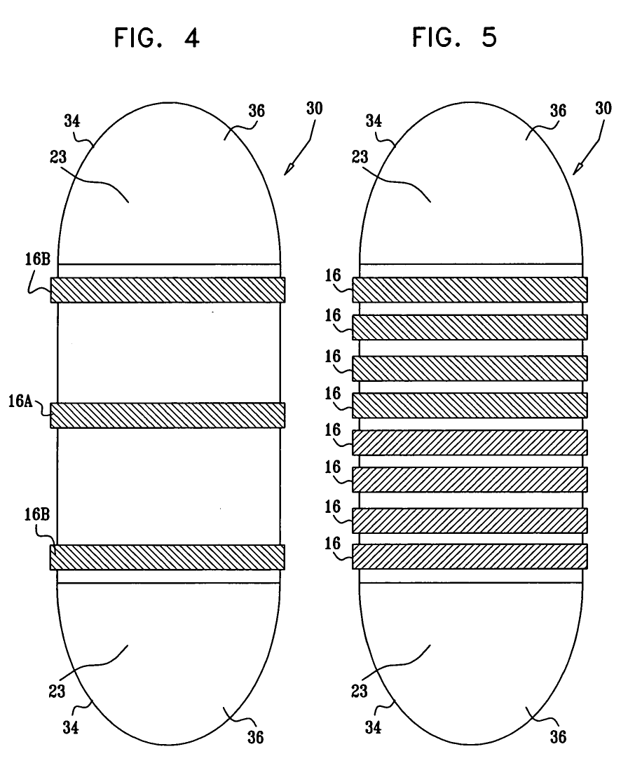

Reference is now made to FIGS. 4 and 5, which illustrate ingestible, electrically-assisted, drug-delivery systems 30, in accordance with embodiments of the present invention. In these embodiments, drug-delivery system 30 comprises a plurality of electrodes 16. For example, in the configuration shown in FIG. 4, system 30 comprises a single cathode 16A and two anodes 16B, or a single anode 16A and two cathodes 16B. Alternatively, as shown in FIG. 5, system 30 comprises a plurality of anodes and cathodes 16.

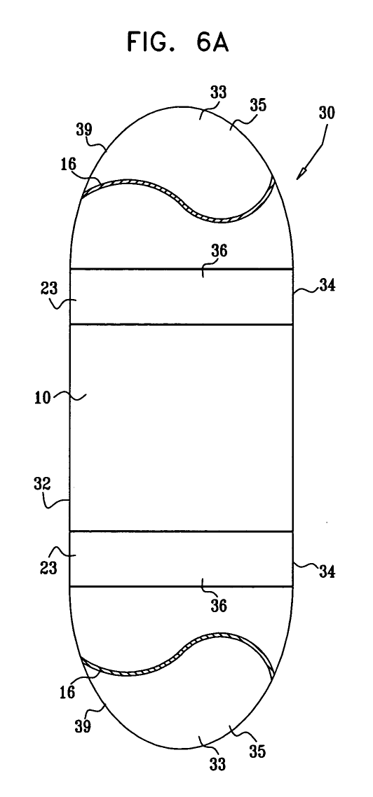

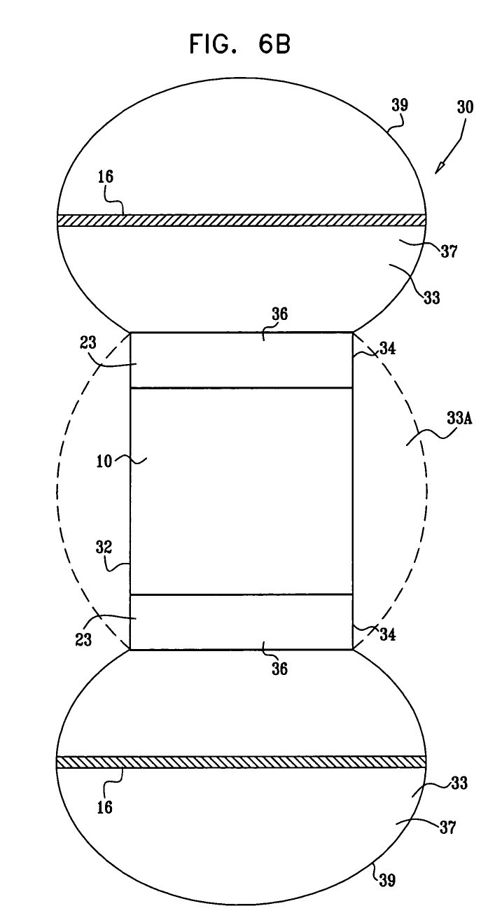

FIGS. 6A and 6B illustrate ingestible, electrically-assisted, drug-delivery system 30 in respective resting and drug-delivery phases thereof, in accordance with an embodiment of the present invention. In this embodiment, device 10 comprises self-expansible portions 33, enclosed in a biologically-inert and biocompatible elastic film 39, such as natural or synthetic thin rubber. For some applications, electrodes 16 are painted on elastic film 39, for better contact between electrodes 16 and the GI walls. The self-expansible effect may be produced, for example, by a chemical reaction of a substance 35 (FIG. 6A), that produces a gas 37, such as CO₂ (FIG. 6B). In the present embodiment, drug-dispensing cavities 23 may be located between self-expansible portions 33 and the main body of device 10. For some applications, system 30 of the present embodiment is used to facilitate contact between electrodes 16 and the GI walls of the colon. For some applications, device 10 comprises a central self-expansible portion 33a, disposed between self-expansible portions 33 that have electrodes 16 thereon. Typically, self-expansible portion 33a is adapted to expand until it contacts the inner wall of the gastrointestinal tract. Thus, self-expansible portion 33a is typically able to expand to at least the same diameter as self-expansible portions 33, and thereby inhibit current flow in the fluid of the lumen of the gastrointestinal tract, and (for constant voltage) facilitate higher current flow in the tissue of the gastrointestinal tract itself. As appropriate, similar central self-expansible portions 33a may be integrated into the embodiments of the invention described with reference to one or more of the other figures of the present patent application.

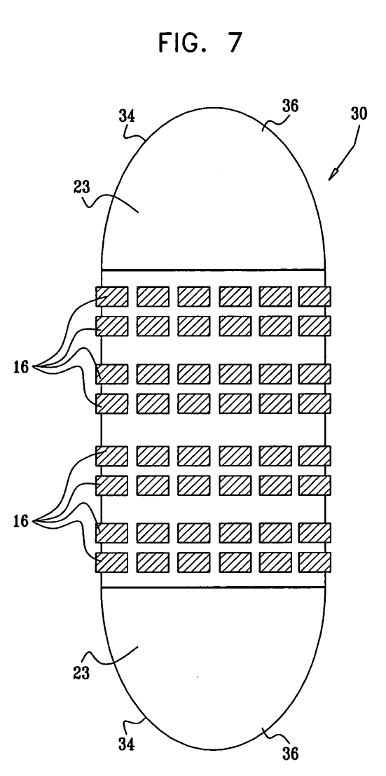

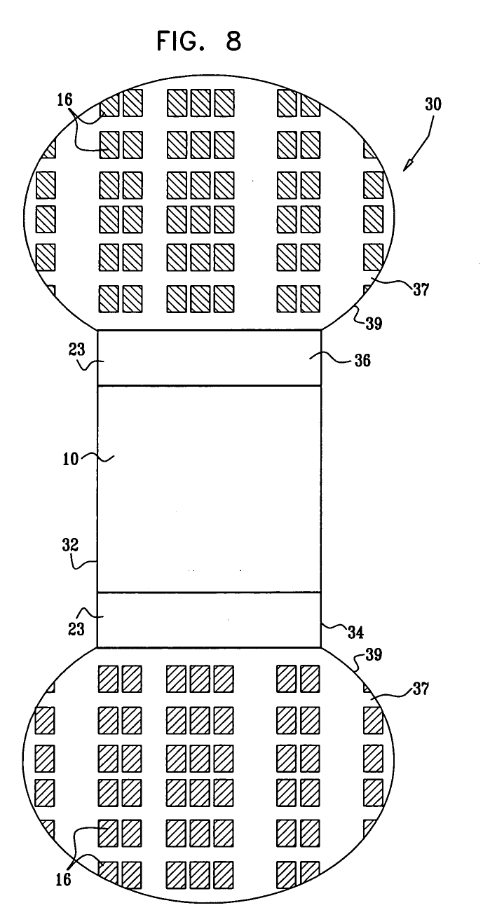

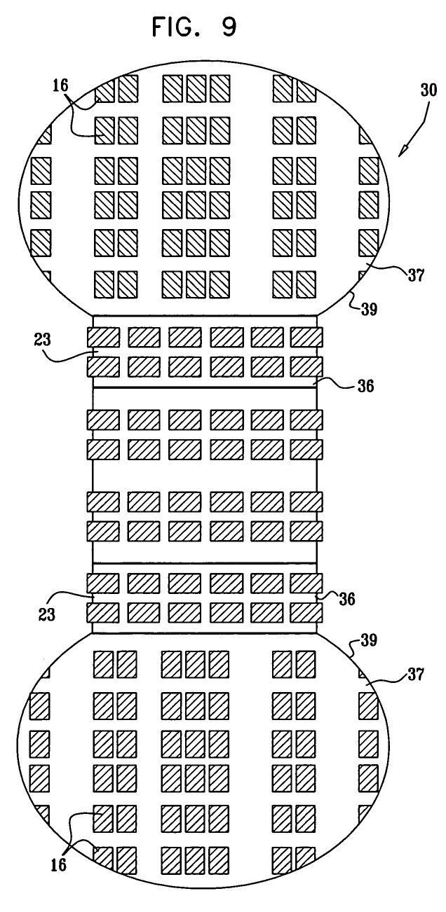

FIGS. 7, 8, and 9 illustrate ingestible, electrically-assisted, drug-delivery systems 30, in accordance with embodiments of the present invention. In these embodiments, system 30 comprises a plurality of electrodes 16 and self-expansible forms.

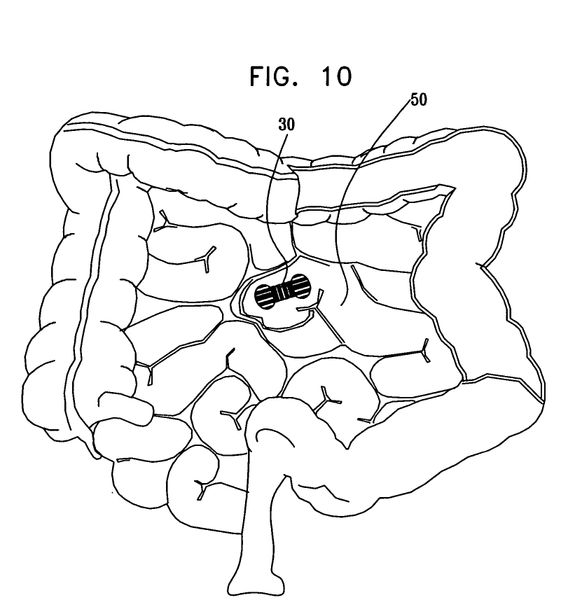

FIG. 10 illustrates ingestible, electrically-assisted, drug-delivery system 30, as it travels in a GI tract 50, in accordance with an embodiment of the present invention. Both the self-expansible portions of system 30 and the plurality of electrodes 16 that cover its exterior are operative to facilitate sliding contact between walls of GI tract 50 and system 30, as suitable for electrostimulation.

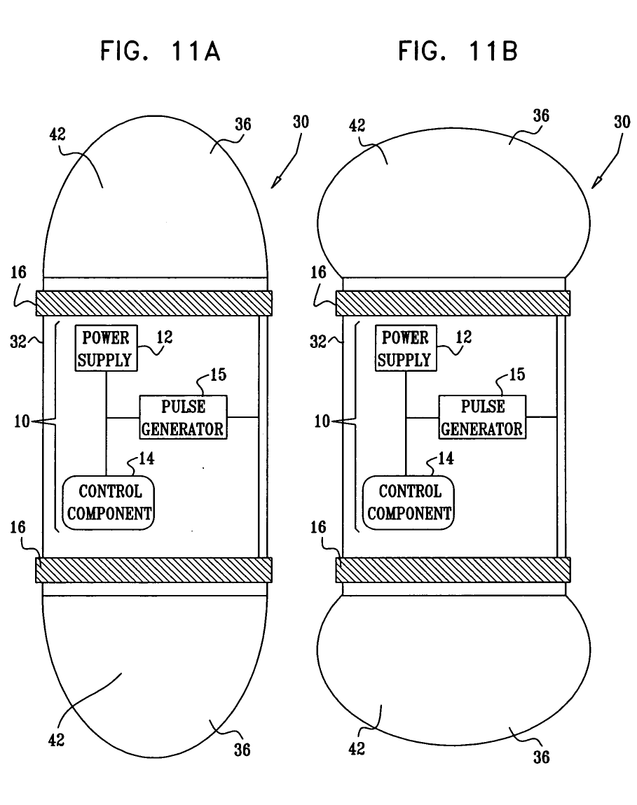

FIGS. 11A-11D illustrate ingestible, electrically-assisted, drug-delivery system 30, in accordance with embodiments of the present invention. In these embodiments, a self-expansible drug matrix is used. Typically, drug 36 is enclosed by a swelling polymer 42, which may be biodegradable, such as hydroxypropylmethylcellulose-HPMC or POLYOX™ (manufactured by The Dow Chemical Company), which expands when brought into contact with GI fluids. Typically, the drug is mixed with the swelling polymer, so as to swell with it.

FIG. 12 illustrates ingestible, electrically-assisted, drug-delivery system 30, formed as a capsule 45, and containing drug 36, as micropellets 43, in accordance with an embodiment of the present invention. A biodegradable film 46 encapsulates micropellets 43. As film 46 disintegrates in the GI tract, drug 36, in the form of micropellets 43, is released.

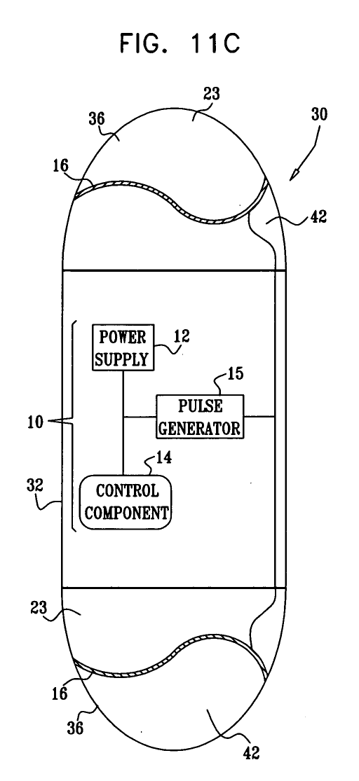

FIG. 13 illustrates ingestible, electrically-assisted, drug-delivery system 30, in accordance with an embodiment of the present invention. In this embodiment, no film is used to contain drug 36. Rather, drug 36 is pressed onto a biocompatible solid bar 48, and slowly dissolves in the GI tract.

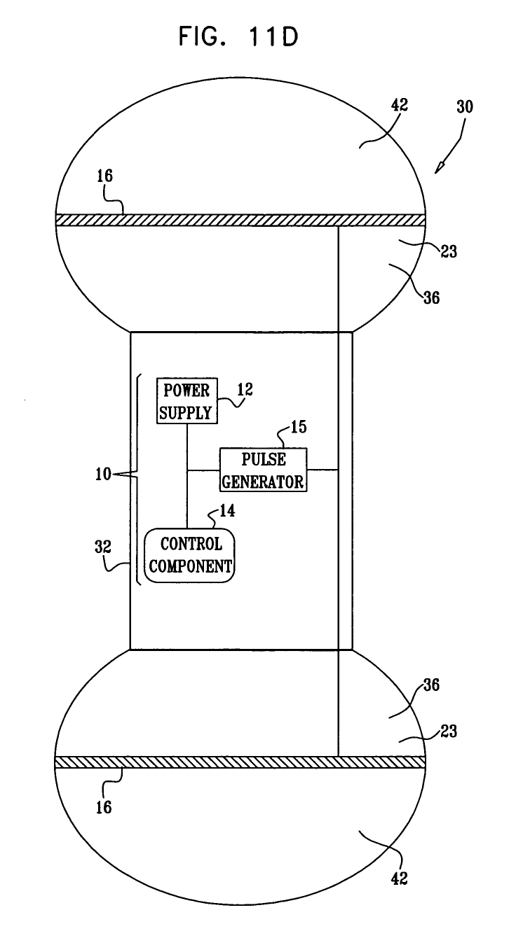

FIGS. 14A and 14B illustrate ingestible, electrically-assisted, drug-delivery system 30 in respective resting and drug-delivery phases thereof, in accordance with an embodiment of the present invention. In this embodiment, drug delivery occurs by osmosis. As a water-soluble plug 29 (FIG. 14A) dissolves, an orifice 38 is opened (FIG. 14B). Uptake of water into drug-dispensing cavity 23 increases the osmotic pressure within the system. The build-up of the osmotic pressure gradient drives the drug through orifice 38 in a controlled manner.

Alternatively, sheath 34 of drug 36 may be formed as cellulose acetate combined with polyethylene glycol (PEG). After ingestion the PEG dissolves, leaving the drug 36 coated with a semi-permeable membrane that controls the release of the drug by osmotic mechanism. Osmognate additives, such as NaCl, added to the drug core, and/or perforation of the sheath 34, may contribute to better controlling the release patterns (osmognates are materials, usually salts, with high solubility and the ability to create high osmotic pressure, to attract water).

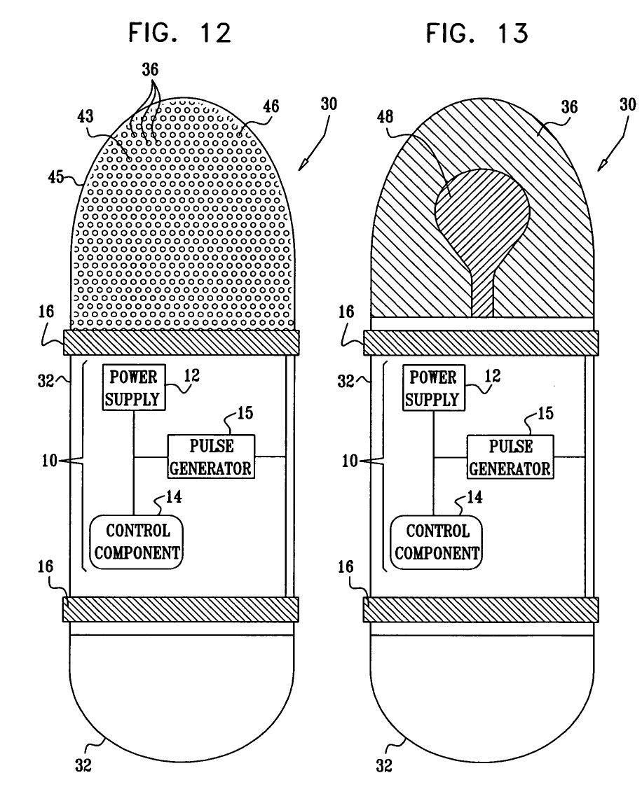

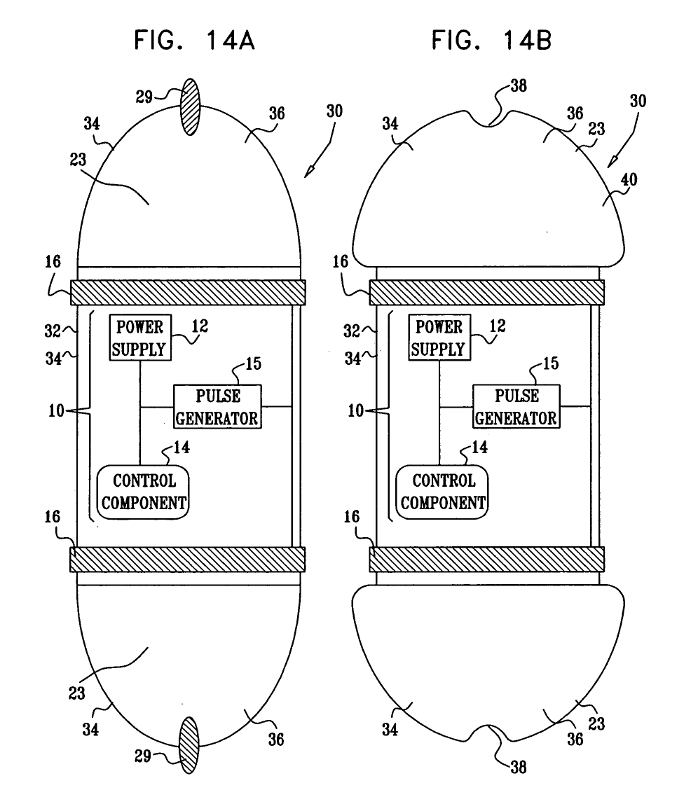

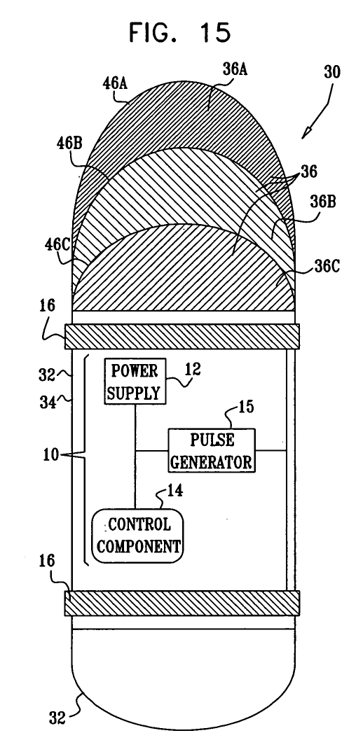

FIG. 15 illustrates ingestible, electrically-assisted, drug-delivery system 30, in accordance with an embodiment of the present invention. In this embodiment, drug release is pH-dependent. Drug 36 is enclosed by at least one film 46A, which dissolves at a specific pH value. For some applications, the pH value is selected to be in the range commonly found in the small intestine, e.g., between about 4.7 and about 6.5, in order to release drug 36 into the small intestine, while substantially preventing the earlier release of the drug in the stomach. Alternatively, the pH is selected to be in the range commonly found in another portion of the GI tract, such as the large intestine. (See Table 1 of the Background Section for exemplary pH values.)

For other applications, the pH value is selected to be in the range commonly found in the stomach, e.g., between about 1.2 and about 3.5, such that film 46A dissolves in the stomach, releasing at least a portion 36A of drug 36. Optionally, system 30 comprises a second film 46B, which dissolves at a pH characteristic of a more distal portion of the GI tract, such as the small intestine, releasing a second portion 36B of drug 36 therein. Further optionally, system 30 comprises a third film 46C, which dissolves at a pH characteristic of a still more distal portion of the GI tract, such as the large intestine (e.g., a pH value of between about 7.5 and about 8.0 for the large intestine), thereby releasing a third portion 36C of drug 36. In this manner, specific drug portions, or even different drugs 36A, 36B, and 36C may be targeted to different portions of the GI tract. Alternatively or additionally, the pH values are selected to release a first portion of drug 36 in the small intestine, and a second portion in the large intestine.

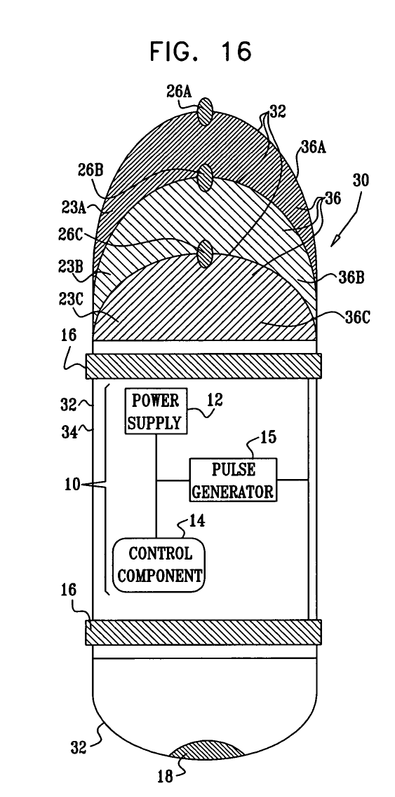

FIG. 16 illustrates ingestible, electrically-assisted, drug-delivery system 30, in accordance with an embodiment of the present invention. In this embodiment, drug release is pH-dependent. Drug 36 is enclosed by housing 32, in two or more drug-dispensing cavities, such as three drug-dispensing cavities 23A, 23B, and 23C, sealed respectively by three electronic valves 26A, 26B, and 26C, the operation of which is controlled by control component 14. A pH sensor 18 typically senses a specific pH value or range of values, and transmits the information to control component 14, which opens one or more of valves 26A, 26B, and 26C, responsive to the sensing.

FIG. 17 illustrates ingestible, electrically-assisted, drug-delivery system 30, in accordance with an embodiment of the present invention. In this embodiment, device 10 comprises ultrasound transducer 22 for providing sonophoresis as a drug transport mechanism. It will be appreciated that sonophoresis may be applied alone, or in combination with electrotransport, using electrodes 16.

FIG. 18 illustrates ingestible, electrically-assisted, drug-delivery system 30, in accordance with an embodiment of the present invention. In this embodiment, device 10 comprises ablation apparatus 24 for providing ablation, such as RF ablation, as a drug transport mechanism. It will be appreciated that ablation may be applied alone, or in combination with electrotransport, using electrodes 16.

Typically, RF ablation parameters include frequencies of about 50 to about 150 kHz, and potentials of about 3-100 volts. These parameters are provided as examples; in accordance with embodiments of the present invention, other parameters, which may be higher or lower, may be used.

Alternatively, ablation apparatus 24 performs microwave ablation, laser ablation, cryogenic ablation, thermal ablation, or liquid jet ablation.

FIG. 19 illustrates ingestible, electrically-assisted, drug-delivery system 30, in accordance with an embodiment of the present invention. In this embodiment, device 10 comprises telemetry system 20, for providing communication with an extracorporeal station 21 (FIG. 2). For example, sensor 18 may transmit extracorporeal station 21 temperature values along the GI tract. These values may be used to inform a person using system 30 of a sudden, or localized temperature increase, suggestive of a problem. Alternatively, sensor 18 may comprise a pH sensor, and extracorporeal station 21 may be used to remotely control valves, such as valves 26A, 26B, and 26C of FIG. 16.

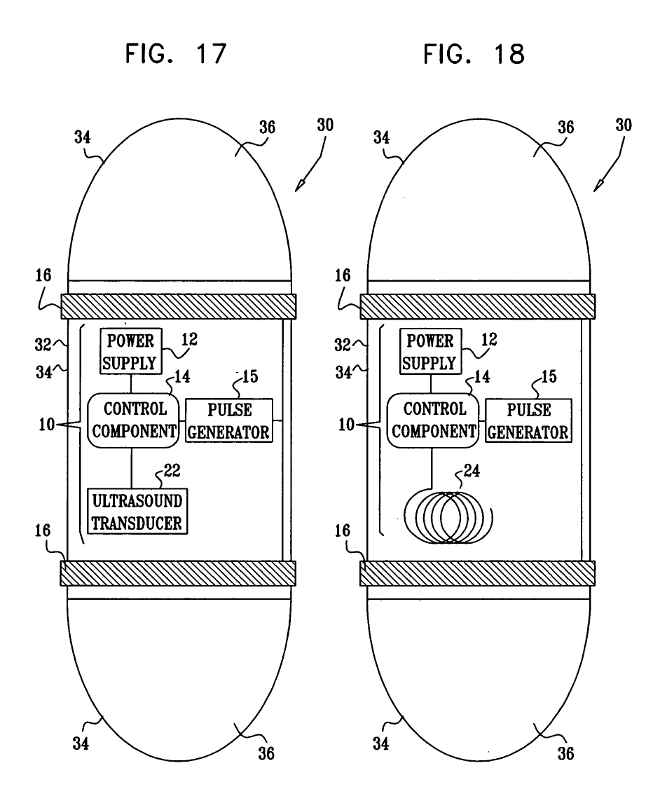

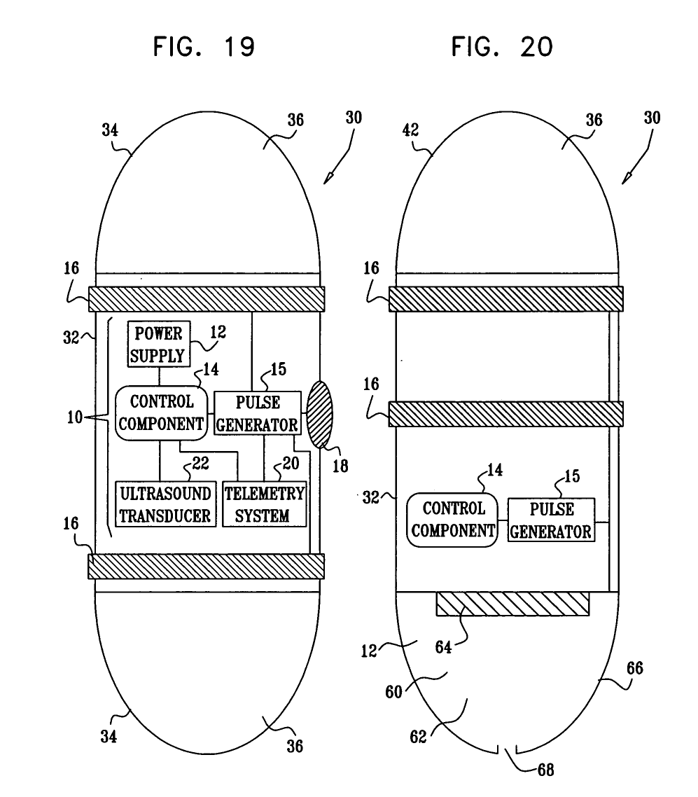

FIG. 20 illustrates ingestible, electrically-assisted, drug-delivery system 30, in accordance with an embodiment of the present invention. In this embodiment, power supply 12 of device 10 is constructed as a galvanic cell 60, comprising an anode 64, a cathode 66, and an orifice 68. As system 30 travels through the GI tract, GI fluids 62 enter galvanic cell 60 via orifice 68, and serve as the electrolyte for the cell.

When the half-life of a drug is less than desired, a controlled release dosage form may be designed, to reduce fluctuation in plasma drug concentration and to provide a more uniform therapeutic effect. Oral controlled-release forms are often designed to maintain therapeutic drug concentrations for at least 12 hours. Several controlled release mechanisms may be used, for example, as taught by Encyclopedia of Controlled Drug Delivery, volume 2, edited by Edith Mathiowitz, pp. 838-841. These are based on the use of specific substances, generally polymers, as a matrix or as a coating. These may be materials that degrade fast or slowly, depending on the desired effect.

In accordance with embodiments of the present invention, drug 36 is released in a controlled manner, using one or more of the following techniques:

The drug, which may be solid, liquid or a suspension in liquid, may be encapsulated in a polymeric material, so that drug release is controlled by diffusion through the capsule walls.

The drug particles may be coated with wax or poorly soluble material, or an insoluble material (e.g., polyvinyl chloride) mixed with a water-soluble, pore forming compound, so that drug release is controlled by the breakdown of the coating.

The drug may be embedded in a slow-release matrix, which may be biodegradable or non-biodegradable, so that the drug release is controlled by diffusion through the matrix, erosion of the matrix, or both.

The drug may be complexed with ion-exchange resins that slow down its release.

The drug may be laminated, as a jellyroll, with a film, such as a polymeric material, which may be biodegradable or nonbiodegradable, so that the drug is released by diffusion, erosion or both.

The drug may be dispersed in a hydrogel, or a substance that forms a hydrogel in the GI tract, so that the drug release is controlled by diffusion of the drug from the water-swollen hydrogel.

Osmotic pressure may be used to release the drug in a controlled manner. Uptake of water into the dosage unit increases the osmotic pressure within the system. The build-up of the osmotic pressure gradient drives the drug through one or more orifices in the dosage form to release the drug in a controlled manner.

The drug may be formed as micropellets, of a density that is lower than that of the GI fluid. The micropellets may float for a long time, before dissolution.

The drug may contain a bioadhesive polymer that adheres to the surface of the epithelium, to extend the time of the drug in the GI tract.

The drug may be chemically bonded to a polymer and released by hydrolysis.

Macromolecular structures of the drug may be formed via ionic or covalent linkages, which control the drug release by hydrolysis, thermodynamic dissociation or microbial degradation.

The drug may be coated with a combination of a soluble and insoluble polymers. When the soluble particles dissolve, they form a microporous layer around the drug core, so that the drug may permeate slowly through the micropores. The rate of release depends on the porosity and thickness of the coating layer. The coating layer components can be varied to prolong release of the drug until the dosage unit is in the presence of a specific pH (e.g., for colon targeting).

The drug may be laminated with a layer designed to dissolve at a specific pH value, for targeting a specific portion of the GI tract.

The drug may be laminated with several layers, each designed to dissolve at a different specific pH value, for targeting different portions of the GI tract, for example, for targeting the colon.

The drug may be designed for pH-independent controlled release, and produced by wet granulating an acidic or basic drug blend with a buffering agent and the appropriate excipients, wherein the granules are then coated with a film, which is permeable in GI fluid and compressed into tablets. Upon oral administration, GI fluid permeates the film coating, and the buffering agents adjust the pH value of the tablet so that the drug can dissolve and permeate out of the dosage form at a constant rate, independent of the pH level in the GI tract.

The drug formulation may be sealed in the insoluble capsule body by means of a water-soluble plug and a hydrogel plug. When the capsule is swallowed, the water-soluble plug dissolves in the gastric juice and exposes the hydrogel plug, which begins to swell. At a predetermined time after ingestion, the hydrogel plug is ejected and the encapsulated drug formation is then released into the alimentary tract.

Alternatively or additionally, other controlled release means known in the art are used.

As appropriate, some or all portions of the capsule are configured to be biodegraded by bacteria in the patient's colon.

It will be appreciated that in accordance with embodiments of the present invention drug release may take any of the following options: controlled release, delayed release, pulsatile release, chronotherapeutic release, immediate release, enterocoated release (activation starts at the small intestine, and the pH-dependent coating protects from the gastric acidic environment). The dosage forms may be chronotherapeutic (adaptation to the circadian rhythm) or colonic delivery type, based on multiple coatings system. The drug may be formed as a capsule of hard gelatin, as compressed powder, or as any other alternative known in the art, for example, hydroxypropyl methylcellulose (HPMC).

When the drug is a peptide formulation or a protein drug, functional additives may be used in order to enable oral delivery. Typical entities are: protease inhibitors, stabilizers, absorption enhancers, and PGP inhibitors, such as verapamil or quinidine.

Additionally, various additives may be used with drug 36. These may include protease inhibitors, which shield against luminal brush, border peptidases, such as Trypsin inhibitor, Chemostatin, Bowman Birk Inhibitor, Aprotinin, SBTI, and polycarbophyl.

Additionally, absorption enhancers, such as NSAIDs, decanoic acid, sodium salicylate, SLS, quaternary ammonium salts, Bile salts-na-cholate, octanoic acid, glycerides, saponins, and/or medium chain fatty acids may be used.

It will be appreciated that in many cases chemical enhancers interact with peptides and proteins. An advantage of some embodiments of the present invention is the ability to circumvent this interaction, by using electrically assisted absorption, in place of chemical enhancers.

Additionally, stabilizers, such as proteins, sugars, polyols, amino acids, inorganic salts, and/or surfactants, may be used.

Furthermore, other pharmaceutically adjuvant for peptides such as buffering agents and/or antioxidants may be used.

Suitable polymers for matrix formation for controlled or slowed release of oral drugs include Acrylates, acrylic acid copolymers, Eudragit, RL/RS type, cellulose derivatives like ethyl cellulose, HPMC, carboxymethylcellulose, carbomers, cellulose acetate, PVA, gums, and any other pharmaceutically acceptable polymers.

In addition to polymers, certain types of lipids may serve as matrix formers as well, for example, glycerol behenate, or glycerol monostearate.

It will be appreciated that the matrix forming polymers may be filled into capsules or compressed into tablets.

Suitable polymers for functional coatings of oral drugs for controlled or slowed drug release include Ethocel (ethyl cellulose), HPMC, Kollicoat (PVA, PVP combinations), CA esters, Eudragits, and enteric coating (pH-dependent) type polymers (Eudragit L,S, CAP, HPMCP, etc.). In addition, acceptable pharmaceutical fillers like MCC, lactose, and ca-phosphate may be used as well.

These coatings may be applied to both tablets and capsules.

It will be appreciated that the type of coating will be determined according to the drug and the desired release profile, such as slow release, enteric (mainly for peptide type), chronotherapeutic, colonic, osmotic, etc.

It will be further appreciated that the coating may be additional to matrix-based dosage forms, either for tablets or for capsules.

Drug candidates for some embodiments of the present invention include peptides, proteins, macromolecules, hormones, polar compounds, and poorly soluble compounds.

Some examples of drugs that may be used as drug 36, in accordance with embodiments of the present invention, include Interleukin 2, TGF-Beta 3, heparin, erythropoietin, cyclosporin, anticancer drugs, viral and non viral vectors for gene delivery, TNF, somatropin, interferones, copaxone, recombinant proteins, immune system modulators, monoclonal antibodies (Herceptin), vaccines, filgastrin, somatostatin, insulins, LHRH antagonists and analogs (Decapeptide, Leuprolide, Goseralin, calcitonin, triptorelin, oxytocin, and sandostatin.

Additionally, small molecule drugs, such as statins, immunosuppressants (e.g., sirolimus, tacrolimus), galantamine, celebrex, and other poorly soluble drugs, or drugs of low availability, may be used. These drugs may be Cox 2 inhibitors, CNS drugs, antibiotics, and any others that require improvement in their oral bioavailability.

Additionally, other known drugs of poor absorption may be used.

Reference is now made to the following examples, which together with the above descriptions illustrate embodiments of the invention in a non-limiting fashion.

### EXAMPLE 1

An electrically assisted, drug-delivery device 10.

Active drug: Insulin.

Filler: microcrystalline cellulose, lactose.

Protease inhibitor: chemostatin, trypsin inhibitor.

The components are mixed and compressed into tablets. An enterocoat is applied to protect from gastric environment. Eudragit L may be used.

### EXAMPLE 2

Similar to Example 1, but additionally including an absorption enhancer, such as decanoic acid.

### EXAMPLE 3

Capsule for oral delivery of copaxone, prepared as in Example 1. The components are dry-mixed and filled into capsules, which are coated with an enterocoat polymer like HPMCP.

### EXAMPLE 4

A tablet for controlled release of cyclosporin.

Both device 10 and HPMC and the drug substance are mixed together, and compressed into tablets (See FIG. 13). The complete system 30 is then coated with ethyl cellulose, which together with the HPMC delays and controls the drug release.

### EXAMPLE 5

An osmotic device. The tablet of Example 4 may be coated with cellulose acetate combined with PEG. After ingestion the PEG dissolves, leaving the tablet coated with a semi-permeable membrane that controls the release of the drug by an osmotic mechanism. Osmognate additives (defined hereinabove), such as NaCl, are added to the drug core, and perforation of the coating may contribute to better controlling the release patterns.

It will be appreciated that any known combination of drug-polymer, dosage form is acceptable, in accordance with embodiments of the present invention.

In accordance with some embodiments of the present invention, the electrically-assisted, drug-delivery system further comprises a visual imaging apparatus, for example, as described in U.S. Pat. No. 5,984,860 to Shan, U.S. Pat. Nos. 5,604,531 and 6,428,469 and U.S. Patent Application 2001/0035902, all to Iddan et al, all of which are incorporated herein by reference

In accordance with some embodiments of the present invention, the electrically-assisted, drug-delivery system further increases the dissolution rate of drugs that dissolve slowly. For example, sonophoresis which produces cavitation has an abrasive effect, and may be operative to enhance the dissolution of drugs of poor solubility.

In accordance with embodiments of the present invention, the electrically-assisted, drug-delivery system is ingestible. Typically, it is free to pass through the GI tract. Alternatively, it may be tethered to a portion of the patient's body, e.g., to a tooth or to a band placed around the patient's head. Alternatively, the electrically-assisted, drug-delivery system may be mounted on a catheter.

Embodiments of the present invention are designed to achieve previously unmet efficiency and bioavailability of orally delivered protein and peptide drugs. It will be appreciated that the electrically-assisted improvement may be performed in addition to and synergistically with known drug enhancers and stabilizers.

FIG. 21A is a schematic illustration of a drug-delivery system 100, comprising a capsule 102, in accordance with an embodiment of the present invention. Capsule 102 comprises a mechanism that is operative to be responsive to its environment, such as, for example, a pH-sensitive coating 104. Coating 104 is typically configured, using techniques known in the art, to dissolve upon entering a small intestine 120 of a patient. In accordance with other embodiments of the present invention described herein and/or shown in the figures, the environmentally-responsive mechanism comprises, for example, a sensor (such as an electronic sensor) a timer, a transmitter/receiver, or a camera.

Capsule 102 typically comprises a drug 106, which is intended for delivery to the patient at a desired site or range of sites in small intestine 120. Drug 106 is typically stored within capsule 102 in a liquid state, although other embodiments of the present invention (some described herein) provide for drug 106 to be stored in another form, such as in solid-, powder-, and/or gel-form.

Capsule 102 typically comprises a driving mechanism 108 located within the capsule (as described hereinbelow) or on an outer surface of the capsule (as described hereinabove). Driving mechanism 108 typically actively drives drug 106 through an orifice 110 of capsule 108 and actively drives the drug through the wall of small intestine 120. For some applications, the orifice is shaped like a nozzle suitable for facilitating passage of a high pressure/high velocity stream. Such nozzles are known, for example, in the field of needleless drug injection.

Typically, the active driving of drug 106 through the GI tract wall is accomplished by: (a) driving the drug through the wall by passage of the drug through tight junctions of the epithelial layer of the small intestine, and/or (b) driving the drug through the wall by penetrating the epithelial cells themselves. Typically, a therapeutically-significant portion of drug 106 is thereby passed into direct contact with the capillary supply of the GI tract (e.g., the small intestine), and therefrom into the systemic circulation. It is noted that this embodiment therefore typically allows entry into the bloodstream of drug molecules which would normally be largely excluded (e.g., due to size or chemical properties).

Depending on the considerations of a given application, orifice 110 may or may not be provided. For example, for electrical active driving mechanisms such as those described hereinabove, drug 106 may be at least in part on an outer surface of capsule 102, or may exit the capsule through an opening larger than orifice 102.

In accordance with an embodiment of the present invention, the dissolving of coating 104 triggers activation of driving mechanism 108, which, in turn, actively drives drug 106 through the wall of small intestine 120. For some applications, coating 104 is configured to dissolve in a pH range of about 4.7-6.5. For example, the coating may be configured to dissolve after about 5-90 minutes of exposure to a pH of about 5 or 6. For these applications, for example, coating 104 may comprise Eudragit or HPMCP, and is typically several tenths of a micron thick.

FIG. 21B is a schematic illustration of drug-delivery system 100, in accordance with another embodiment of the present invention. In this embodiment, coating 104 is applied at a first thickness over a first portion of capsule 102 (e.g., over driving mechanism 108), and at a second thickness over a second portion of capsule 102 (e.g., over orifice 110). As particularly shown in the embodiment of FIG. 21B, no coating 104 (or, alternatively, only a small amount of coating 104) is applied to the second portion of capsule 102. Alternatively or additionally, different types of coatings are applied to different portions of capsule 102, e.g., in order to provide for the respective portions of the capsule to be exposed to the small intestine at different times.

In an embodiment, a sealing mechanism, such as a plug ill, is placed within orifice 110, and is automatically removed therefrom upon activation of driving mechanism 108. For example, driving mechanism 108 may generate pressure within capsule 102, which ejects plug 111 and facilitates rapid ejection of drug 106 through the wall of small intestine 120. Although plug 111 is not shown in all of the figures, it is to be appreciated that it may be integrated in any of the gas generation embodiments or other embodiments described herein, as appropriate.

It is noted that a sealing mechanism as described may be provided in combination with or separately from a variable-thickness coating 104.

It is further noted that although the figures generally show a small space between the outer surface of capsule 102 and the surrounding small intestinal wall, this is for purposes of visual clarity only. In practice, the intestinal wall is typically in contact with the capsule throughout the capsule's peristaltically-driven passage through the GI tract.

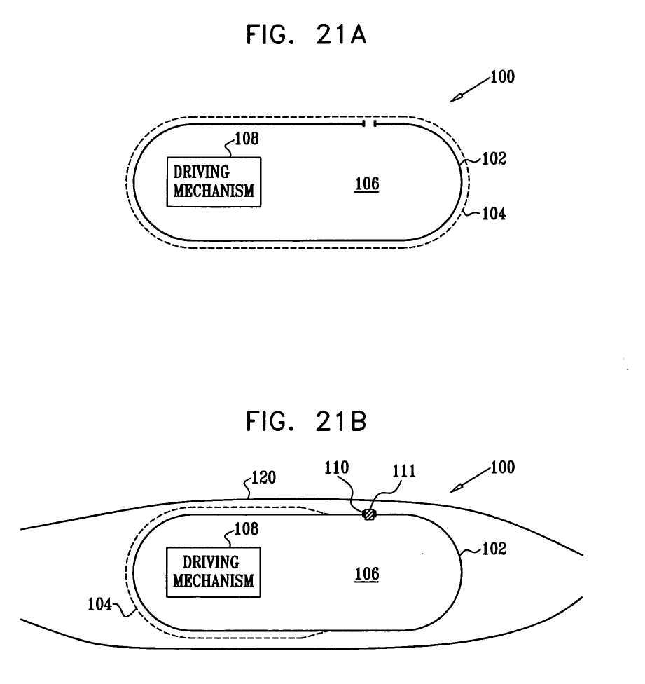

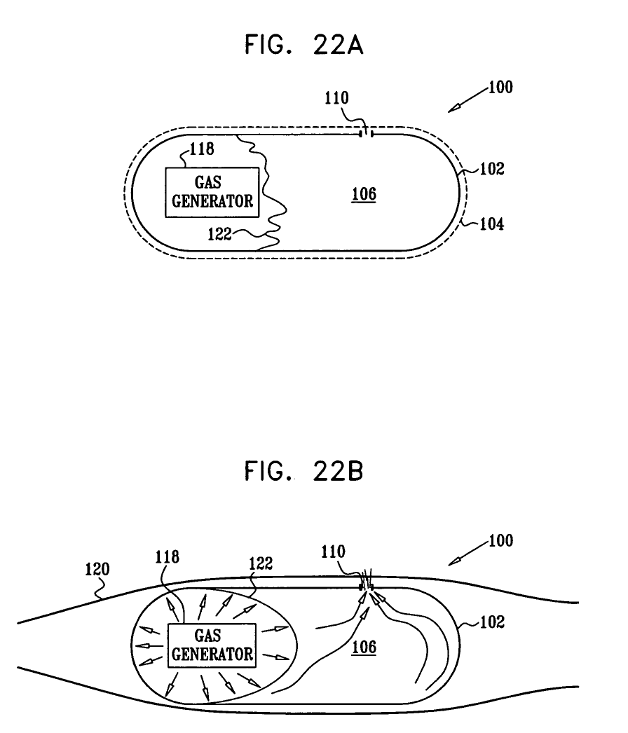

FIG. 22A is a schematic illustration of drug-delivery system 100, in accordance with an embodiment of the present invention. In this embodiment, driving mechanism 108 comprises a gas generator 118 and a movable member, such as a membrane 122. Membrane 122 moves within capsule 102 in response to the generation of gas by generator 118. In other configurations (not shown), the movable member comprises a piston. In yet other configurations (not shown), a movable member is not provided, but instead gas generator 118 acts directly on drug 106.

FIG. 22B is a schematic illustration of drug-delivery system 100, configured as in the embodiment of FIG. 22A, but in a drug-delivery phase thereof. In this embodiment, the dissolving of coating 104 activates gas generator 118 to release a gas that deflects membrane 122. This deflection, in turn, applies pressure to drug 106, driving it out of orifice 110, through the epithelial layer of small intestine 120, and into contact with the capillary circulation of the small intestine.

For these embodiments, as well as other described hereinbelow, orifice 110 typically has a characteristic diameter between about 20 and 400 microns. As appropriate for a given application, the diameter may be between about 20-50 microns, 50-150 microns, or 150-400 microns. The pressure developed within the capsule typically increases by about 0.1 to 5 atmospheres, for example, about 0.5 to 1.5 atmospheres. For some applications, the pressure change occurs over a time period of less than about 1 minute (e.g., for gas-generating reactions between chemicals) . For other applications (e.g., those utilizing electrolysis), the pressure change typically occurs over longer time periods, such as 1-20 minutes, or 20-60 minutes. For applications in which the increase in pressure occurs over more than about 1 second or several seconds, a plug such as plug 111 is typically but not necessarily utilized. As appropriate, characteristics of system 100 may be selected so as to utilize the technology of needleless injection.

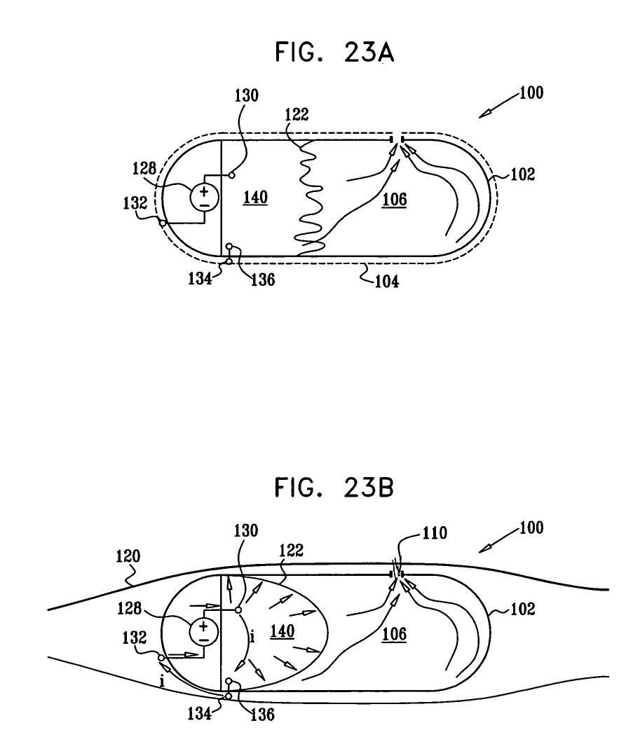

Reference is now made to FIGS. 23A and 23B, which are schematic illustrations of drug-delivery system 100 in respective resting and drug-delivery phases thereof, in accordance with an embodiment of the present invention. In this embodiment, gas generator 118 comprises a power source, such as a battery 128, having positive and negative poles thereof coupled to respective electrodes 130 and 132. (Alternatively, the positive and negative poles are reversed.)

Electrode 130 is typically in contact with a liquid, such as a saline solution 140 contained within capsule 102. Solution 140, in turn, is typically in contact with or otherwise mechanically coupled to membrane 122. Although battery 128 may be placed within solution 140, it is typically in a separate compartment, with a barrier 138 protecting the battery from the solution.

Electrode 132 is typically mounted to an external surface of capsule 102, within coating 104. Electrode 132 may protrude from the surface of the capsule (as shown), or, alternatively, may be flat mounted on the outer surface of the capsule.

In addition, capsule 102 comprises an electrode having a first electrode contact 136 electrically coupled to solution 140, and a second electrode contact 134 mounted to the outer surface of capsule 102. In some configurations, electrode contacts 134 and 136 are embodied as opposite faces of a flat metallic surface which forms a portion of the casing of capsule 102.

In this embodiment, coating 104 typically has very low electrical conductivity, and can be generally considered to act as an electrical insulator. Thus, when coating 104 is still present (e.g., before ingestion, and while capsule 102 is in the patient's stomach), the current drain from battery 128 is minimal or essentially zero.

As shown in FIG. 23B, after entry of capsule 102 into the small intestine and upon the dissolving of coating 104, electrode 132 and electrode contact 134 are electrically coupled via the ion-rich fluids naturally present in the small intestine. A current is thereby able to flow, powered by battery 128, from electrode 130 via solution 140 to electrode contact 136. The flow of the current through solution 140 is associated with electrolysis of the water, and generates a gas. The gas generated by this process deflects membrane 122 and forces drug 106 out of orifice 110, as described hereinabove.

For some applications, battery 128 comprises biocompatible/biodegradable components, such as zinc and manganese dioxide.

In an embodiment, instead of or in addition to battery 128, capsule 102 comprises a piezoelectric crystal coupled to a capacitor. The capacitor stores energy applied to capsule 102 during gastric contractions and during the initial stages of peristalsis in the small intestine.

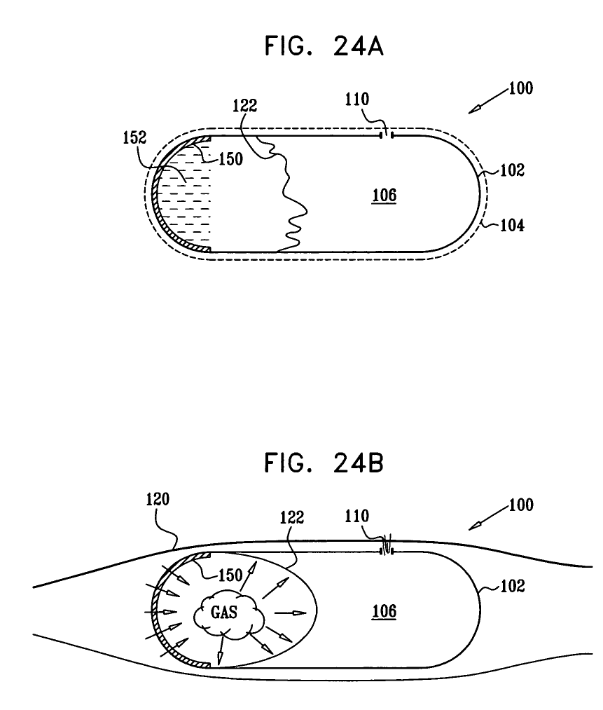

Reference is now made to FIGS. 24A and 24B, which are schematic illustrations of drug-delivery system 100 in respective resting and drug-delivery phases thereof, in accordance with an embodiment of the present invention. In this embodiment, gas generator 118 comprises a hydrophilic membrane 150 and a substance 152 typically adjacent to the hydrophilic membrane. Hydrophilic membrane 150 is typically embedded in or otherwise coupled to the outer surface of capsule 102, on an opposite side of membrane 122 relative to that side of membrane 122 that faces drug 106. For some applications, a suitable membrane may be obtained from Celgard, Inc. (Charlotte, N.C.).

Substance 152 is typically disposed within capsule 102, and has the characteristic of rapidly releasing gas upon contact with the fluid of the GI tract. For some applications, substance 152 comprises sodium bicarbonate.

Hydrophilic membrane 150 is protected from the fluid of the GI tract by membrane 104 until capsule 102 arrives at a suitable region of the GI tract, such as a portion of the small intestine having a particular pH. At this point, membrane 104 dissolves, and hydrophilic membrane 150 allows passage of the GI tract fluid into the capsule, where it contacts substance 152. As shown in FIG. 24B, gas is released rapidly in response to the reaction of the GI tract fluid with substance 152. In turn, membrane 122 is deflected and drug 106 is ejected at high pressure and velocity through orifice 110 and through the wall of the small intestine.

By virtue of the physical properties of hydrophilic membrane 150, the fluid which passes through hydrophilic membrane 150 to react with substance 152 also wets the hydrophilic membrane. The wetting of hydrophilic membrane 152 typically provides a partial or substantially-complete barrier to the release of gas through the hydrophilic membrane, and thereby facilitates the desired mechanical work performed by the generated gas on membrane 122 and drug 106.

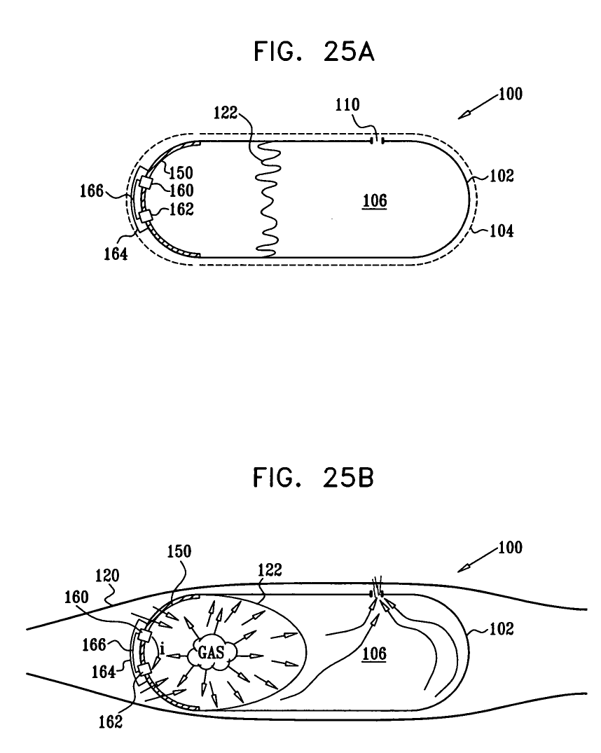

Reference is now made to FIGS. 25A and 25B, which are schematic illustrations of drug-delivery system 100 in respective resting and drug-delivery phases thereof, in accordance with an embodiment of the present invention. In this embodiment, gas generator 118 comprises hydrophilic membrane 150, as described hereinabove, and two electrodes 160 and 162 typically but not necessarily embedded in the casing of capsule 102. Electrodes 160 and 162 typically comprise different metals, such as zinc and manganese dioxide, or zinc and silver oxide. A conductor 166 electrically couples electrode 160 to electrode 162. Typically, conductor 166 and electrodes 160 and 162 are encased within an insulator 164, and, in combination, constitute a galvanic cell.

As shown in FIG. 25B, after coating 104 dissolves in response to the pH of the small intestine, fluid from the GI tract enters capsule 102 via hydrophilic membrane 150. The fluid, once inside the capsule, provides (a) a low resistance pathway for current flow between electrodes 160 and 162, and, in parallel, (b) the water source for electrolysis and corresponding rapid production of gas. The released gas, as described hereinabove, deflects membrane 122 and drives drug 106 out of orifice 110 and through the intestinal wall.

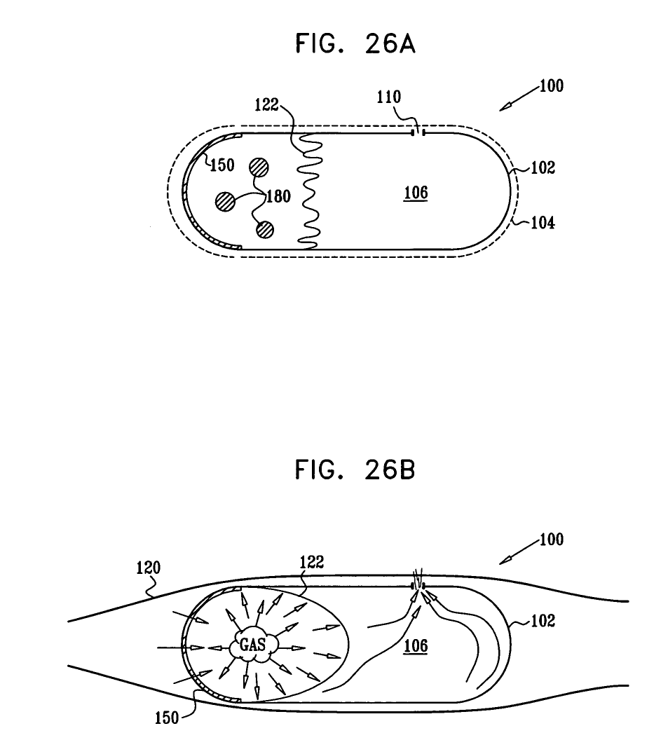

Reference is now made to FIGS. 26A and 26B, which are schematic illustrations of drug-delivery system 100 in respective resting and drug-delivery phases thereof, in accordance with an embodiment of the present invention. In this embodiment, gas generator 118 comprises hydrophilic membrane 150 and one or more gas-releasing elements 180. For some applications, gas-releasing elements 180 comprise elemental sodium or elemental calcium, which reacts with the acidic GI tract fluid passing through hydrophilic membrane 150 after the dissolving of coating 104. As shown in FIG. 26B, this reaction rapidly releases gas, and drives membrane 122 to push drug 106 through orifice 110 and through the epithelial layer of the small intestine.

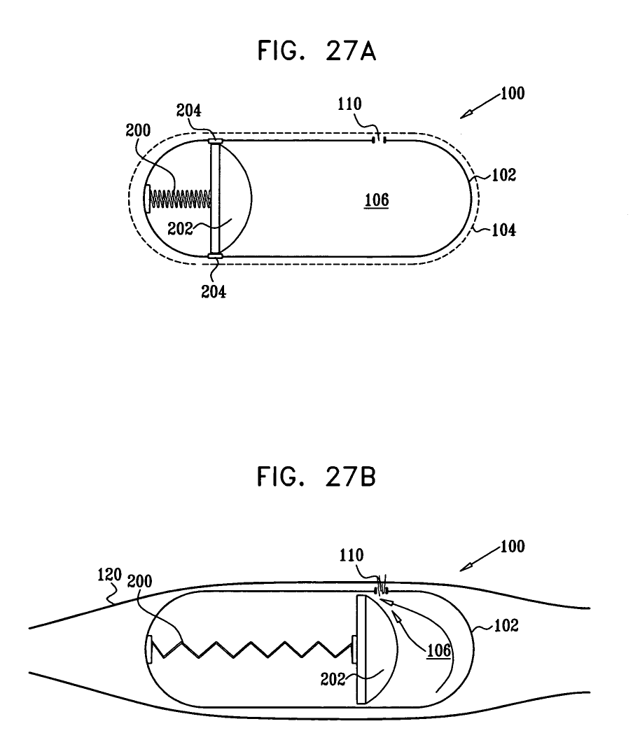

Reference is now made to FIGS. 27A and 27B, which are schematic illustrations of drug-delivery system 100 in respective resting and drug-delivery phases thereof, in accordance with an embodiment of the present invention. In this embodiment, driving mechanism 108 (FIG. 21A) comprises a piston 202 and a piston driver 200. For some applications, piston driver 200 comprises a mechanical spring, as shown. For other applications, piston driver 200 comprises a source of compressed air.

In accordance with this embodiment of the present invention, capsule 202 is typically stored with piston driver 200 in the tense state. The piston driver is prevented from releasing its energy by a portion 204 of coating 104 that is disposed in a position within capsule 102 that inhibits motion of piston 202. In an application, portion 204 is an extension of coating 104, and comprises the same material as coating 104. In another application, portion 204 comprises a pH-sensitive adhesive that exhibits sufficiently-strong adhesive properties, prior to digestion, whereby it is able to hold piston 202 in place. After ingestion of the capsule and the dissolving of coating 104 in the small intestine, portion 204 is exposed to the acidic environment of the small intestine, and dissolves as well, thereby freeing piston 202.

As shown in FIG. 27B, after portion 204 releases piston 202, piston driver 200 drives piston 202 to force drug 106 through orifice 110 and through the wall of the small intestine.

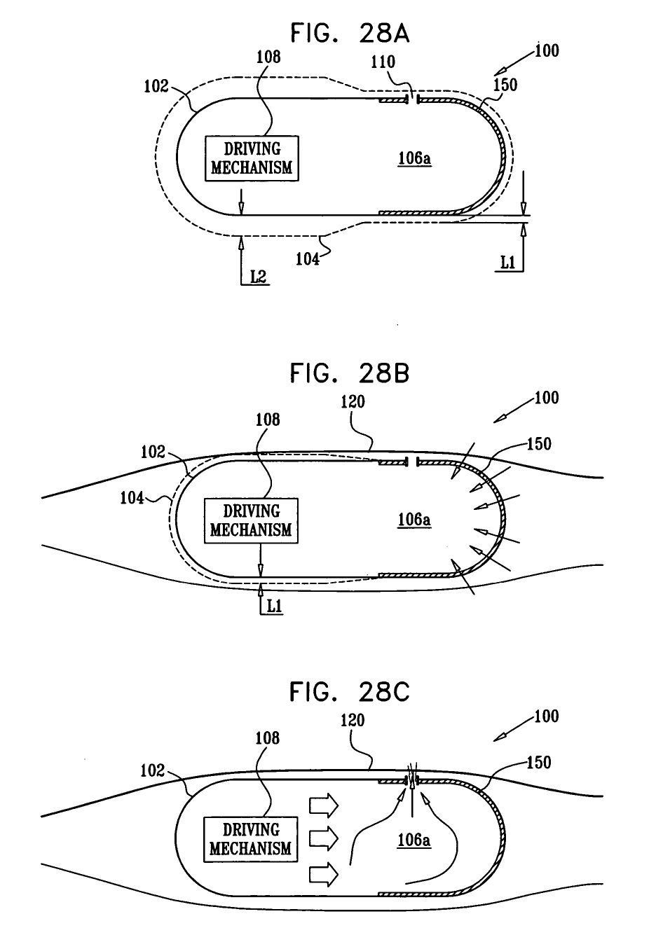

Reference is now made to FIGS. 28A, 28B, and 28C, which are schematic illustrations of drug-delivery system 100 in respective resting, partially-activated, and drug-delivery phases thereof, in accordance with an embodiment of the present invention. In this embodiment, capsule 102 comprises a drug 106a (such as drug 106), stored in powder form within capsule 102. A hydrophilic membrane 150, in addition to any uses it may have in activating driving mechanism 108 as described hereinabove, allows fluid from the GI tract to mix with drug 106a. Typically, capsule 102 is configured to facilitate this mixing prior to activation of driving mechanism 108. In an embodiment, this pre-mixing of drug 106a with the GI tract fluid is brought about by setting a thickness L1 of coating 104 to be lower in a region surrounding hydrophilic membrane 150 than a thickness L2 of coating 104 in a region surrounding driving mechanism 108.

In this manner, as shown in FIG. 28B, pH-sensitive coating 104 over hydrophilic membrane 150 essentially completely dissolves, allowing the GI tract fluid to enter the capsule and mix with drug 106a. During this process, the portion of coating 104 over driving mechanism 108 is smaller than as shown in FIG. 28A, but not yet sufficiently small to cause the activation of the driving mechanism.

Subsequently, as shown in FIG. 28C, the portion of coating 104 over driving mechanism 108 also dissolves, causing the activation of the driving mechanism. This activation causes (now substantially liquefied) drug 106a to be (a) driven out of orifice 110 and (b) driven through the wall of the small intestine by the mechanical force applied thereto by driving mechanism 108.

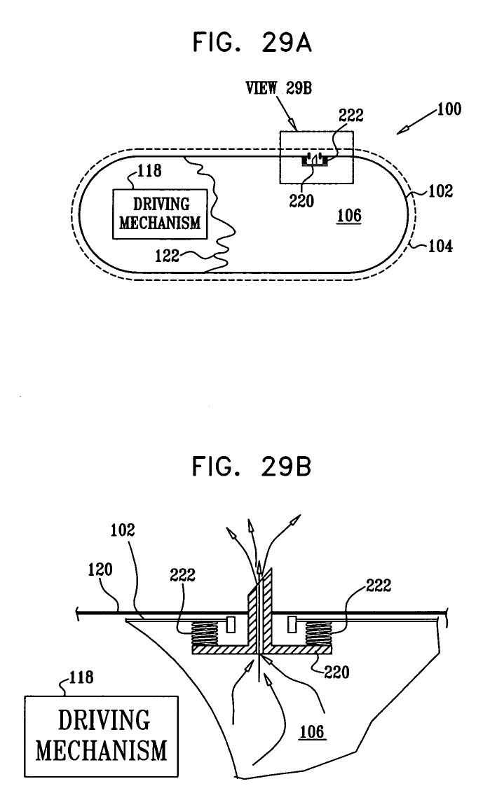

Reference is now made to FIGS. 29A and 29B, which are schematic illustrations of drug-delivery system 100 in respective resting and drug-delivery phases thereof, in accordance with an embodiment of the present invention. For clarity of illustration, FIG. 29B shows an expanded view of the portion of system 100 shown in FIG. 29A.

In this embodiment, capsule 102 comprises a hollow needle 220 located adjacent to orifice 110 and in communication with drug 106. In the resting phase, as shown in FIG. 29A, one or more elastic elements 222 hold hollow needle 220 generally within capsule 102, such that the sharp tip of the needle does not extend past coating 104, and, typically, does not extend past the outer surface of the capsule. As appropriate, elastic elements 222 may comprise springs, spring-like mechanical elements, or compressed air.

Upon activation of driving mechanism 108, as shown in FIG. 29B, a substantial force is generated by drug 106 upon needle 220. This force surpasses the force generated by elastic elements 222, and thrusts hollow needle 220 out of the body of capsule 102 and through the wall of the small intestine. While the pressure within capsule 102 is still high, drug 106 passes through the channel in hollow needle 220, past the endothelial layer of the small intestine, and into contact with the underlying capillary bed. When the high pressure subsequently declines (e.g., after several seconds to about a minute), the force provided by elastic elements 222 surpasses that generated by driving mechanism 108, and hollow needle 220 retracts within the body of capsule 102.

For some applications, needle 220 is not hollow, but instead provides a transient small hole in the wall of the small intestine through which drug 106 may pass.

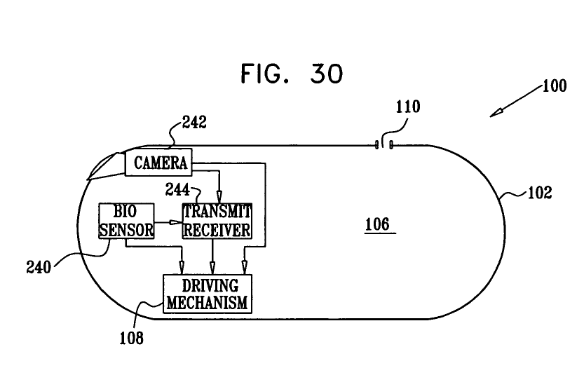

FIG. 30 is a schematic illustration of drug-delivery system 100, in accordance with another embodiment of the present invention. In this embodiment, the functionality for activating driving mechanism 108, described hereinabove as being provided by coating 104, is supplemented or replaced by other activating functionalities.

For some applications, capsule 102 comprises a bio-sensor 240 that detects a biological or physiological parameter, and activates driving mechanism 108 responsive thereto. As appropriate, bio-sensor 240 may comprise one or more of the following:

(a) an enzymatic sensor, selectively sensitive to an enzyme indicative of the capsule's presence in a given portion of the GI tract and/or sensitive to a pathological condition, such as inflammation or GI bleeding,

(b) a temperature sensor, e.g., a sensor sensitive to elevated temperatures associated with inflammation,

(c) a pH sensor, e.g., a pH sensor sensitive to a particular pH in the range of about 4.7-6.5,

(d) a timer, typically comprising chemicals that react in a known manner to activate driving mechanism 108 at a predetermined time following an event such as the patient squeezing the capsule or the patient ingesting the capsule.

Alternatively or additionally, capsule 102 comprises a camera 242, such as is produced by Given Imaging, Ltd. (Israel), which records an image of the GI tract for on-board analysis and, if appropriate, activation of driving mechanism 108 in response to the image.

For some applications, capsule 102 comprises a transmit/receive unit 244, adapted to transmit a signal responsive to an image recorded by the camera and/or responsive to a reading by- bio-sensor 240. The transmitted data are typically analyzed in real-time, and a decision is made (e.g., by a physician or by a computer external to the patient) whether and when to administer drug 106.

As will be apparent to one of ordinary skill in the art having read the present patent application, it is also possible to configure capsule 102 to control the quantity of drug 106 administered. For example, drug 106 may be stored in several chambers within capsule 102, and the signal sent to the transmit/receive unit instructs the driving mechanism to deliver the drug from none, one, some, or all of the chambers.

For some applications, techniques described hereinabove are practiced in combination with techniques described in one or more of the articles, patents and/or patent applications mentioned hereinabove. By way of example and not limitation, embodiments of the present invention comprising a piston or spring may use spring-release techniques described in one or more of these patents or patent applications.

It is expected that during the life of this patent many relevant drugs will be developed and the scope of the term drug is intended to include all such new technologies a priori.

As used herein the term “about” refers to +/−10%.

In the description hereinabove of embodiments of the invention, various oral dosage forms are described, for example, capsules and tablets. In the claims, the word “capsule” is to be understood to refer to oral dosage forms generally, i.e., comprising capsules, tablets, and similar forms, for example, as shown in FIGS. 3-20 with respect to drug-delivery system 30, or as shown in FIGS. 21-30 with respect to capsule 102.

As used in the context of the present patent application and in the claims, the word “drug” means any natural or synthetic chemical that may be administered as an aid in the diagnosis, treatment, cure, mitigation, or prevention of disease or other abnormal conditions, or to improve health.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. Apparatus for drug administration, comprising:
an ingestible capsule, which comprises:
a drug, stored by the capsule;
an environmentally-sensitive mechanism, adapted to change a state thereof responsive to a disposition of the capsule within a gastrointestinal tract of a subject; and
a driving mechanism, which, in response to a change of state of the environmentally-sensitive mechanism, is adapted to drive the drug directly through an endothelial layer of the gastrointestinal tract.

2. The apparatus according to claim 1, wherein the drug is stored in the capsule in liquid form.

3. The apparatus according to claim 1, wherein the environmentally-sensitive mechanism is adapted to undergo the change of state when the capsule is in a small intestine of the subject.

4. The apparatus according to claim 1, wherein the environmentally-sensitive mechanism is adapted to undergo the change of state when the capsule is in a large intestine of the subject.

5. The apparatus according to claim 1, wherein the environmentally-sensitive mechanism is adapted to undergo the change of state when the capsule is in a stomach of the subject.

6. The apparatus according to claim 1, wherein the environmentally-sensitive mechanism is essentially entirely biodegradable.

7. The apparatus according to claim 1, wherein the driving mechanism is essentially entirely biodegradable.

8. The apparatus according to claim 1,
wherein the environmentally-sensitive mechanism comprises a sensor adapted to sense an indication of a distance traveled by the capsule in the gastrointestinal tract, and
wherein the environmentally-sensitive mechanism is adapted to undergo the change of state responsive to the distance.

9. The apparatus according to claim 8, wherein the sensor comprises an inertial sensor.

10. The apparatus according to claim 1, wherein at least 80% of the mass of the capsule is biodegradable.

11. The apparatus according to claim 10, wherein at least 95% of the mass of the capsule is biodegradable.

12. The apparatus according to claim 11, wherein essentially the entire capsule is biodegradable.

13. The apparatus according to claim 1, wherein the capsule comprises a self-expansible portion, which is adapted to expand responsive to the change of state of the environmentally-sensitive mechanism.

14. The apparatus according to claim 13, wherein a characteristic diameter of the self-expansible portion is adapted to increase by at least 100%, responsive to the change of state of the environmentally-sensitive mechanism.

15. The apparatus according to claim 13, wherein the self-expansible portion is adapted to expand responsive to expansion of a gas within the self-expansible portion.

16. The apparatus according to claim 13, wherein the self-expansible portion is adapted to expand responsive to an inflow of fluid from the gastrointestinal tract.

17. The apparatus according to claim 13,
wherein a characteristic diameter of the self-expansible portion immediately prior to expanding is smaller than a characteristic diameter of a portion of the gastrointestinal tract containing the capsule, and
wherein a characteristic diameter of the self-expansible portion following expanding is at least as large as a characteristic diameter of the portion of the gastrointestinal tract containing the capsule.

18. The apparatus according to claim 13,
wherein the capsule comprises an electrode on an outer surface of the self-expansible portion, and
wherein the driving mechanism is adapted to drive current through the electrode when the self-expansible portion is in an expanded state thereof.

19. The apparatus according to claim 18,
wherein the self-expansible portion includes a first self-expansible portion, at a first end of the capsule,
wherein the capsule includes a second self-expansible portion, at a second end of the capsule, and
wherein the capsule comprises an electrode on an outer surface of the second self-expansible portion.

20. The apparatus according to claim 19, wherein the capsule comprises a third self-expansible portion, disposed between the first and second self-expansible portions.

21. The apparatus according to claim 20, wherein the capsule comprises an electrode on an outer surface of the third self-expansible portion.

22. The apparatus according to claim 20, wherein the capsule contains no electrodes on an outer surface of the third self-expansible portion.

23. The apparatus according to claim 1, wherein the environmentally-sensitive mechanism comprises a coating on a surface of the capsule.

24. The apparatus according to claim 23, wherein the coating comprises a pH-sensitive coating.

25. The apparatus according to claim 24, wherein the pH-sensitive coating is sensitive to a pH that is characteristic of a small intestine.

26. The apparatus according to claim 23,
wherein the coating is adapted to cover a portion of the driving mechanism, prior to the change of state, in a manner that substantially prevents contact of the driving mechanism with a first fluid of the gastrointestinal tract, and
wherein the coating is adapted to uncover the portion of the driving mechanism in response to the coating contacting a second fluid of the gastrointestinal tract.

27. The apparatus according to claim 26, wherein the driving mechanism is adapted to drive the drug directly through the endothelial layer of the gastrointestinal tract responsive to uncovering of the portion of the driving mechanism.

28. The apparatus according to claim 1, wherein the environmentally-sensitive mechanism comprises a timer, adapted to change the state of the environmentally-sensitive mechanism responsive to a duration of the capsule in the gastrointestinal tract.

29. The apparatus according to claim 28, wherein the timer comprises an electronic timer.

30. The apparatus according to claim 28, wherein the timer comprises a chemical timer, adapted to change the state of the environmentally-sensitive mechanism responsive to a chemical reaction.

31. The apparatus according to claim 1, wherein the environmentally-sensitive mechanism comprises a camera, adapted to image the gastrointestinal tract, and wherein the driving mechanism is adapted to drive the drug through the endothelial layer in response to an image acquired by the camera.

32. The apparatus according to claim 31, wherein the capsule comprises a control component, adapted to interpret the acquired image and activate the driving mechanism responsive thereto.

33. The apparatus according to claim 31, wherein the capsule comprises a transmit/receive unit, adapted to transmit data responsive to the acquired image, to receive an instruction responsive to the transmission, and to activate the driving mechanism responsive to the instruction.

34. The apparatus according to claim 1, wherein the environmentally-sensitive mechanism comprises a sensor, adapted to sense a characteristic of the gastrointestinal tract, and wherein the driving mechanism is adapted to drive the drug through the endothelial layer in response to the sensed characteristic.

35. The apparatus according to claim 34, wherein the capsule comprises a control component, adapted to interpret the sensed characteristic and activate the driving mechanism responsive thereto.

36. The apparatus according to claim 34, wherein the capsule comprises a transmit/receive unit, adapted to transmit data responsive to the sensed characteristic, to receive an instruction responsive to the transmission, and to activate the driving mechanism responsive to the instruction.

37. The apparatus according to claim 34, wherein the sensor comprises an enzymatic sensor.

38. The apparatus according to claim 34, wherein the sensor comprises an optical sensor.

39. The apparatus according to claim 34, wherein the sensor comprises a thermal sensor.

40. The apparatus according to claim 34, wherein the sensor comprises a pH sensor.

41. The apparatus according to claim 40, wherein the pH sensor is adapted to detect a pH between about 4.7 and about 6.5.

42. The apparatus according to claim 40, wherein the pH sensor is adapted to detect a pH between about 1.2 and about 3.5.

43. The apparatus according to claim 40, wherein the pH sensor is adapted to detect a pH between about 4.6 and about 6.0.

44. The apparatus according to claim 40, wherein the pH sensor is adapted to detect a pH between about 7.5 and about 8.0.

45. The apparatus according to claim 34, wherein the sensor comprises a sensor adapted to detect a pathological condition of the gastrointestinal tract.

46. The apparatus according to claim 45, wherein the sensor comprises a sensor adapted to detect bleeding in the gastrointestinal tract.

47. The apparatus according to claim 45, wherein the sensor comprises a sensor adapted to detect inflammation in the gastrointestinal tract.

48. The apparatus according to claim 1,
wherein the capsule comprises a needle comprising a sharp tip thereof, and
wherein the tip of the needle is adapted to contact the endothelial layer of the gastrointestinal tract in response to the change of state of the environmentally-sensitive mechanism.

49. The apparatus according to claim 48, wherein the needle is hollow.

50. The apparatus according to claim 48, wherein the needle is not hollow.

51. The apparatus according to claim 48,
wherein the capsule comprises an elastic element, adapted to maintain the sharp tip of the needle at an original position that is substantially within the capsule, prior to the change of state,
wherein, in response to an action of the driving mechanism, the elastic element is adapted to change shape in a manner that permits the sharp tip of the needle to contact the endothelial layer of the gastrointestinal tract, and
wherein, at a time after initiation of the driving of the drug through the endothelial layer, the elastic element is adapted to cause the sharp tip of the needle to withdraw to the original position.

52. The apparatus according to claim 48, wherein the driving mechanism is adapted to drive the needle to puncture the endothelial layer of the gastrointestinal tract at a puncture site, in response to the change of state of the environmentally-sensitive mechanism.

53. The apparatus according to claim 52, wherein the driving mechanism is adapted to drive the drug through the puncture site.

54. The apparatus according to claim 1, wherein the drug is stored in the capsule in powder form.

55. The apparatus according to claim 54, wherein the capsule is adapted to mix the drug in powder form with a fluid, in response to the change of state of the environmentally-sensitive mechanism.

56. The apparatus according to claim 55,
wherein the fluid includes fluid of the gastrointestinal tract, and
wherein the capsule is adapted to mix the drug in powder form with the gastrointestinal tract fluid, in response to the change of state of the environmentally-sensitive mechanism.

57. The apparatus according to claim 55,
wherein the fluid comprises fluid stored within the capsule, separately from the drug in powder form, and
wherein the capsule is adapted to mix the drug in powder form with the fluid stored within the capsule, in response to the change of state of the environmentally-sensitive mechanism.

58. The apparatus according to claim 1,
wherein the driving mechanism comprises a control component, a first electrode, a second electrode, and a third electrode,
wherein the control component is adapted to drive an iontophoretic current between the first and second electrodes, and
wherein the control component is adapted to drive an electropulsation current through the third electrode.

59. The apparatus according to claim 1,
wherein the driving mechanism comprises a control component, a first electrode, and a second electrode, and
wherein the control component is adapted to drive a current between the first and second electrodes in response to the change of state of the environmentally-sensitive mechanism.

60. The apparatus according to claim 59, wherein the environmentally-sensitive mechanism comprises a coating on a surface of the capsule.

61. The apparatus according to claim 59, wherein the driving mechanism comprises the first and second electrodes and no other electrodes.

62. The apparatus according to claim 59, wherein the driving mechanism comprises more than three electrodes.

63. The apparatus according to claim 59, wherein the control component is adapted to configure the current to ablate at least a portion of the endothelial layer of the gastrointestinal tract.

64. The apparatus according to claim 59, wherein the control component comprises a battery.

65. The apparatus according to claim 64, wherein the battery is biodegradable.

66. The apparatus according to claim 64, wherein the battery comprises zinc and manganese dioxide.

67. The apparatus according to claim 59, wherein the driving mechanism comprises a third electrode, and wherein the control component is adapted to drive a current between the first and third electrodes in response to the change of state of the environmentally-sensitive mechanism.

68. The apparatus according to claim 67, wherein the first electrode is physically disposed on the capsule between the second electrode and the third electrode.

69. The apparatus according to claim 67, wherein the control component is adapted to configure the current driven between the first and second electrodes to be substantially identical to the current driven between the first and third electrodes.

70. The apparatus according to claim 67,
wherein the control component is adapted to configure the current driven between the first and second electrodes to consist essentially of an iontophoretic current, and
wherein the control component is adapted to configure the current driven between the first and third electrodes to consist essentially of an electropulsation current.

71. The apparatus according to claim 59, wherein the control component is adapted to drive the current between the first and second electrodes at a level sufficient to iontophoretically drive the drug through the endothelial layer of the gastrointestinal tract.

72. The apparatus according to claim 71, wherein the control component is adapted to configure a voltage drop between the first and second electrodes to be less than about 3 volts.

73. The apparatus according to claim 71, wherein the control component is adapted to configure the current to be substantially DC.

74. The apparatus according to claim 71, wherein the control component is adapted to configure the current to have a characteristic frequency less than about 50 Hz.

75. The apparatus according to claim 74, wherein the control component is adapted to configure the current to have a characteristic frequency less than about 5 Hz.

76. The apparatus according to claim 71, wherein the control component is adapted to configure the current to have an amplitude less than about 5 mA.

77. The apparatus according to claim 76, wherein the control component is adapted to configure the current to have an amplitude greater than about 0.5 mA.

78. The apparatus according to claim 59, wherein the control component is adapted to configure the current to increase conduction of the drug through tight junctions of the endothelial layer of the gastrointestinal tract by means of electropulsation.

79. The apparatus according to claim 78, wherein the control component is adapted to configure a voltage drop between the first and second electrodes to be between about 3 and about 12 volts.

80. The apparatus according to claim 78, wherein the control component is adapted to configure a voltage drop between the first and second electrodes to be between about 12 and about 50 volts.

81. The apparatus according to claim 78, wherein the control component is adapted to configure the current to have a characteristic frequency less than about 300 Hz.

82. The apparatus according to claim 81, wherein the control component is adapted to configure the current to have a characteristic frequency less than about 100 Hz.

83. The apparatus according to claim 82, wherein the control component is adapted to configure the current to have a characteristic frequency greater than about 1 Hz.

84. The apparatus according to claim 83, wherein the control component is adapted to configure the current to have a characteristic frequency greater than about 10 Hz.

85. The apparatus according to claim 82, wherein the control component is adapted to configure the current to have a characteristic frequency less than about 20 Hz.

86. The apparatus according to claim 85, wherein the control component is adapted to configure the current to have a characteristic frequency greater than about 10 Hz.

87. The apparatus according to claim 59, wherein the control component is adapted to configure the current to: (a) be at a level sufficient to iontophoretically drive the drug through the endothelial layer of the gastrointestinal tract, and (b) increase conduction of the drug through tight junctions of the endothelial layer of the gastrointestinal tract by means of electropulsation.

88. The apparatus according to claim 87,
wherein the current includes an iontophoretic current and an electropulsation current,
wherein the control component is adapted to drive the iontophoretic current between the first and second electrodes, and
wherein the control component is adapted to drive the electropulsation current between the first and second electrodes.

89. The apparatus according to claim 87, wherein the control component is adapted to configure the current to have a high-frequency component and a low-frequency component.

90. The apparatus according to claim 89, wherein the control component is adapted to configure the high-frequency component and the low-frequency component to have frequencies that are respectively greater than and less than 5 Hz.

91. The apparatus according to claim 89, wherein the control component is adapted to drive the high-frequency component and the low-frequency component at the same time.

92. The apparatus according to claim 89, wherein the control component is adapted to drive the high-frequency component prior to driving the low-frequency component.

93. The apparatus according to claim 92, wherein the control component is adapted to initiate driving the high-frequency component at least 30 seconds prior to driving the low-frequency component.

94. The apparatus according to claim 1, wherein the driving mechanism comprises a piston and a piston driver, and wherein the piston driver is adapted to drive the piston to drive the drug from the capsule.

95. The apparatus according to claim 94, wherein the piston driver comprises a compressed gas that is adapted to expand in response to the change of state of the environmentally-sensitive mechanism.

96. The apparatus according to claim 94, wherein the piston driver comprises a spring-like mechanical element.

97. The apparatus according to claim 1, wherein the driving mechanism comprises a gas generator, which, in response to the change of state of the environmentally-sensitive mechanism, is adapted to generate a gas which on expansion thereof performs work on the drug in a manner that drives the drug from the capsule and directly through the endothelial layer of the gastrointestinal tract.

98. The apparatus according to claim 97, wherein the gas generator is adapted to generate, within about 1 minute, a pressure change of at least 0.2 atmosphere within the capsule, in response to the change of state of the environmentally-sensitive mechanism.

99. The apparatus according to claim 97, wherein the gas generator is adapted to generate, within about 20 minutes, a pressure change of at least 0.2 atmosphere within the capsule, in response to the change of state of the environmentally-sensitive mechanism.

100. The apparatus according to claim 97,
wherein the capsule comprises a flexible membrane between the gas generator and the drug,
wherein the membrane is adapted to be deflected in response to the generation of the gas, and
wherein the membrane, in response to being deflected, is adapted to drive the drug through the endothelial layer of the gastrointestinal tract.

101. The apparatus according to claim 97, wherein the gas generator is in a common compartment with the drug, and wherein the gas generated by the gas generator, in direct contact with the drug, drives the drug from the capsule and directly through the endothelial layer of the gastrointestinal tract.

102. The apparatus according to claim 97, wherein the gas generator is adapted to generate a pressure change of at least about 0.1 atmosphere within the capsule, in response to the change of state of the environmentally-sensitive mechanism.

103. The apparatus according to claim 102, wherein the gas generator is adapted to configure the pressure change to be less than about 5 atmospheres, in response to the change of state of the environmentally-sensitive mechanism.

104. The apparatus according to claim 103, wherein the gas generator is adapted to configure the pressure change to be between about 0.5 and 3 atmospheres, in response to the change of state of the environmentally-sensitive mechanism.

105. The apparatus according to claim 103, wherein the gas generator is adapted to configure the pressure change to occur during less than about 1 minute.

106. The apparatus according to claim 103, wherein the gas generator is adapted to configure the pressure change to occur over a time period having a duration between about 1 and 10 minutes.

107. The apparatus according to claim 103, wherein the gas generator is adapted to configure the pressure change to occur over a time period having a duration between about 10 and 120 minutes.

108. The apparatus according to claim 97, wherein the gas generator is adapted to facilitate entry into the capsule of fluid of the gastrointestinal tract in response to the change of state of the environmentally-sensitive mechanism, and to generate the gas responsive to the entry of the gastrointestinal tract fluid into the capsule.

109. The apparatus according to claim 108,
wherein the gas generator comprises a substance, and
wherein the gas generator is adapted to generate the gas by causing contact of the gastrointestinal tract fluid with the substance, in response to the change of state of the environmentally-sensitive mechanism.

110. The apparatus according to claim 109, wherein the substance comprises a substance selected from the list consisting of elemental sodium and elemental calcium.

111. The apparatus according to claim 97,
wherein the gas generator comprises a substance having a pH greater than 7, and
wherein the gas generator is adapted to generate the gas by facilitating contact between the substance and fluid of the gastrointestinal tract, in response to the change of state of the environmentally-sensitive mechanism.

112. The apparatus according to claim 111, wherein the substance comprises sodium bicarbonate.

113. The apparatus according to claim 111, wherein the gas generator comprises a membrane proximate the substance, which is adapted to facilitate entry of the gastrointestinal tract fluid into the capsule, through the membrane, in response to the change of state of the environmentally-sensitive mechanism.

114. The apparatus according to claim 113, wherein the membrane comprises a hydrophilic membrane.

115. The apparatus according to claim 113, wherein the membrane is integral to an outer surface of the capsule.

116. The apparatus according to claim 97, wherein the gas generator comprises a galvanic cell.

117. The apparatus according to claim 116, wherein the galvanic cell comprises a first electrode comprising zinc and a second electrode comprising manganese dioxide.

118. The apparatus according to claim 116, wherein the galvanic cell comprises first and second galvanic cell electrodes, which are adapted to pass current through fluid of the gastrointestinal tract at a level sufficient to electrolyze the fluid and generate the gas.

119. The apparatus according to claim 116, wherein the gas generator comprises a membrane, which is adapted to facilitate entry of fluid of the gastrointestinal tract into the capsule, through the membrane, and into contact with the first and second galvanic cell electrodes, in response to the change of state of the environmentally-sensitive mechanism.

120. The apparatus according to claim 97,
wherein an outer surface of the capsule is shaped so as to define an orifice having an edge, the edge of the orifice generally being in contact with a portion of the gastrointestinal tract at a time after the environmentally-sensitive mechanism changes state, and
wherein the gas generator and the drug are disposed within the capsule in such a manner that the generation of the gas drives the drug through the orifice and, therefrom, through the portion of the gastrointestinal tract.

121. The apparatus according to claim 120, wherein the capsule comprises a seal, which blocks the orifice prior to the change of state of the environmentally-sensitive mechanism, and which is adapted to be removed from the orifice in response to the generation of the gas by the gas generator.

122. The apparatus according to claim 121, wherein the seal comprises a plug, adapted to:
be disposed within the orifice prior to the change of state of the environmentally-sensitive mechanism,
resist ejection from the orifice during an initial rise in pressure within the capsule that occurs in response to the generation of the gas by the gas generator, and
be ejected from the orifice when the pressure within the capsule surpasses a threshold pressure.

123. The apparatus according to claim 120, wherein the capsule is shaped such that a characteristic diameter of the orifice is between about 20 and about 400 microns.

124. The apparatus according to claim 123, wherein the capsule is shaped such that the characteristic diameter of the orifice is between about 20 and about 50 microns.

125. The apparatus according to claim 123, wherein the capsule is shaped such that the characteristic diameter of the orifice is between about 50 and about 300 microns.

126. The apparatus according to claim 97, wherein the gas generator comprises an electrical power source, adapted to drive current through a fluid in a manner that causes the generation of the gas by electrolysis of the fluid.

127. The apparatus according to claim 126,
wherein the power source comprises first and second poles,
wherein the gas generator comprises the fluid,
wherein the first pole of the power source is directly electrically coupled to the fluid,
wherein the gas generator comprises a coupling electrode, electrically coupled to the second pole of the power source,
wherein the gas generator comprises a second electrode, electrically coupled via the fluid to the first pole of the power source, and substantially electrically isolated from the coupling electrode prior to the change of state of the environmentally-sensitive mechanism, and
wherein the environmentally-sensitive mechanism is adapted, in response to the change of state, to establish electrical contact between the coupling electrode and the second electrode.

128. The apparatus according to claim 126, wherein the fluid includes fluid of the gastrointestinal tract, and wherein the gas generator is adapted, in response to the change of state of the environmentally-sensitive mechanism, to drive the current through the fluid of the gastrointestinal tract.

129. Apparatus for administration of a drug, comprising:
an ingestible capsule adapted to store the drug, the capsule comprising:
an environmentally-sensitive mechanism, adapted to change a state thereof responsive to a disposition of the capsule within a gastrointestinal tract of a subject; and
a driving mechanism, which, in response to a change of state of the environmentally-sensitive mechanism, is adapted to drive the drug directly through an endothelial layer of the gastrointestinal tract.

130. Apparatus for administration of a drug, comprising:
an ingestible environmentally-sensitive mechanism, adapted to change a state thereof responsive to a disposition thereof within a gastrointestinal tract of a subject; and
a driving mechanism, which, in response to a change of state of the environmentally-sensitive mechanism, is adapted to drive the drug directly through an endothelial layer of the gastrointestinal tract.

131. Apparatus, comprising:
a capsule adapted to travel through a gastrointestinal tract of a subject, the capsule comprising:
first and second electrodes; and
a control component, adapted to drive, at each of a plurality of sites longitudinally distributed along the gastrointestinal tract, an iontophoretic current that travels from the first electrode, through an endothelial layer of the gastrointestinal tract, and to the second electrode.

132. The apparatus according to claim 131, wherein the control component is adapted to drive the iontophoretic current while the capsule is in motion.

133. The apparatus according to claim 131, wherein the control component is adapted to configure a voltage drop between the first and second electrodes to be less than about 3 volts, and to configure a characteristic frequency of the iontophoretic current to be less than about 5 Hz.

134. The apparatus according to claim 131, wherein the capsule comprises a self-expansible portion, and wherein the first electrode is disposed on an outer surface of the self-expansible portion.

135. The apparatus according to claim 134, wherein the capsule comprises a second self-expansible portion, and wherein the second electrode is disposed on an outer surface of the second self-expansible portion.

136. The apparatus according to claim 134, wherein the capsule comprises a coating on an outer surface thereof, and wherein the control component is adapted to initiate driving the iontophoretic current in response to a change of state of the coating.

137. Apparatus, comprising:
a capsule adapted to travel through a gastrointestinal tract of a subject, the capsule comprising:
first and second electrodes; and
a control component, adapted to drive, at each of a plurality of sites longitudinally distributed along the gastrointestinal tract, an electropulsation current that travels from the first electrode, through an endothelial layer of the gastrointestinal tract, and to the second electrode.

138. The apparatus according to claim 137, wherein the control component is adapted to drive the electropulsation current while the capsule is in motion.

139. The apparatus according to claim 137, wherein the control component is adapted to configure a voltage drop between the first and second electrodes to be greater than about 3 volts, and to configure a characteristic frequency of the electropulsation current to be between about 1 and 30 Hz.

140. The apparatus according to claim 137, wherein the capsule comprises a self-expansible portion, and wherein the first electrode is disposed on an outer surface of the self-expansible portion.

141. The apparatus according to claim 140, wherein the capsule comprises a second self-expansible portion, and wherein the second electrode is disposed on an outer surface of the second self-expansible portion.

142. The apparatus according to claim 140, wherein the capsule comprises a coating on an outer surface thereof, and wherein the control component is adapted to initiate driving the electropulsation current in response to a change of state of the coating.

143. Apparatus, comprising:
a capsule adapted to travel through a gastrointestinal tract of a subject, the capsule comprising:
first and second electrodes;
a coating on an outer surface of the capsule; and
a control component, adapted to drive an iontophoretic current that travels from the first electrode, through an endothelial layer of the gastrointestinal tract, and to the second electrode, in response to a change of state of the coating.

144. The apparatus according to claim 143, wherein the capsule comprises first and second self-expansible portions, at respective ends of the capsule, and wherein the first and second electrodes are disposed on respective outer surfaces of the first and second self-expansible portions.

145. A method for administration of a drug, comprising:
administering to a subject an ingestible capsule that includes a drug;
detecting a disposition of the capsule within a gastrointestinal tract of the subject; and
in response to detecting the disposition, driving the drug directly through an endothelial layer of the gastrointestinal tract.

146. The method according to claim 145, wherein driving the drug comprises iontophoretically driving the drug.

147. The method according to claim 145, wherein driving the drug comprises applying an electropulsation current configured to facilitate the driving of the drug.

148. The method according to claim 145, wherein driving the drug comprises expanding a portion of the capsule in response to detecting the disposition.

149. The method according to claim 145, wherein detecting the disposition comprises causing an interaction between a coating on an outer surface of the capsule and fluid of the gastrointestinal tract.

150. An electrically assisted, drug-delivery system, comprising:
a biologically inert and biologically compatible device, comprising:
a power supply;
a control component, in power communication with said power supply; and
at least one apparatus for electrically assisted drug transport, said apparatus being in signal communication with said control component and in power communication with said power supply; and
a drug attached to said device.

151. The system of claim 150, wherein said drug further includes pharmaceutically acceptable additives for absorption enhancement.

152. The system of claim 150, wherein said drug further includes pharmaceutically acceptable additives for improved bioavailability.

153. The system of claim 150, wherein said drug further includes pharmaceutically acceptable additives for controlled release.

154. The system of claim 150, wherein said drug further includes pharmaceutically acceptable additives for ph-dependent controlled release.

155. The system of claim 150, wherein said drug further includes pharmaceutically acceptable additives for time-dependent controlled release.

156. The system of claim 150, wherein said apparatus for electrically assisted drug transport, comprises an apparatus for at least two electrotransport processes.

157. The system of claim 150, wherein said at least one apparatus for electrically assisted drug transport comprises an apparatus for sonophoresis.

158. The system of claim 150, wherein said at least one apparatus for electrically assisted drug transport comprises an apparatus for at least one ablation process.

159. The system of claim 150, wherein said at least one apparatus for electrically assisted drug transport comprises an apparatus for at least two processes, selected from the group consisting of electrotransport, sonophoresis, and ablation.

160. The system of claim 150, wherein said device includes at least one self-expansible portion, for making good contact with the gastrointestinal walls.

161. The system of claim 150, wherein said power supply is a galvanic cell, which uses gastrointestinal fluids as an electrolyte.

162. The system of claim 150, wherein said device includes a pH sensor.

163. The system of claim 150, wherein said device includes a telemetry system for communicating with an extracorporeal station.

164. The system of claim 150, wherein said device is ingestible.

165. The system of claim 150, wherein said device is attached to a catheter.

166. The system of claim 150, wherein said device further includes an imaging apparatus.

167. The system of claim 150, wherein said at least one apparatus for electrically assisted drug transport comprises an apparatus for at least one electrotransport process.

168. The system of claim 167, wherein said apparatus for electrotransport is further operative to enhance peristalsis, by electrostimulation.

169. The system of claim 150, wherein said device further defines a drug-dispensing cavity.

170. The system of claim 169, wherein said drug-dispensing cavity is adapted for controlled release.

171. The system of claim 169, wherein said drug-dispensing cavity is adapted for pH dependent controlled release.

172. The system of claim 169, wherein said drug-dispensing cavity is self-expansible, to make better contact with the gastrointestinal walls.

173. A method of oral drug delivery, comprising:
orally inserting a drug into the gastrointestinal tract; and
inducing transport through the gastrointestinal walls, by a method selected from the group consisting of: at least one electrotransport process, sonophoresis, and at least one ablation process.
